(19) United States
(12) Patent Application Publication
(10) Pub. No.: US 2003/0219818 A1
(43) Pub. Date: Nov. 27, 2003

(54) Methods and compositions for determining neoplastic disease responsiveness to antibody therapy

(76) Inventors: Sean P. Bohen, Mountain View, CA (US); Ronald Levy, Stanford, CA (US); David Botstein, Belmont, CA (US); Patrick O. Brown, Stanford, CA (US)

Correspondence Address: BOZICEVIC, FIELD & FRANCIS LLP, 200 MIDDLEFIELD RD, SUITE 200, MENLO PARK, CA, 94025, US

(21) Appl. No.: 10/435,508

(22) Filed: May 9, 2003

Related U.S. Application Data
(60) Provisional application No. 60/379,847, filed on May 10, 2002.

(51) Int. Cl. C12Q 1/68; G01N 33/574

(52) U.S. Cl. 435/6; 435/7.23

(57) ABSTRACT

Methods are provided for determining whether a subject suffering from a neoplastic condition, e.g., non-Hodgkin's lymphoma (NHL), such as follicular lymphoma, is responsive to antineoplastic therapy, such as antibody therapy, e.g., Rituximab. In practicing the subject methods, an expression profile is obtained from the subject suffering from NHL and employed to determine whether the subject is responsive to antineoplastic therapy. In addition, reagents and kits thereof that find use in practicing the subject methods are provided.

### CROSS-REFERENCE TO RELATED APPLICATIONS

This application claims priority (pursuant to 35 U.S.C. §119 (e)) to the filing date of the U.S. Provisional Patent Application Serial No. 60/379,847 filed May 10, 2002; the disclosure of which is herein incorporated by reference.

### INTRODUCTION

1. Field of the Invention

The field of this invention is non-Hodgkin's lymphoma and the treatment therapy thereof, particularly using antibody therapeutics, e.g., rituximab.

2. Background of the Invention

In the fall of 1997, the anti-CD20 monoclonal antibody, rituximab (currently sold under the brand name RITUXAN®), was approved for the treatment of refractory or relapsed low-grade B-cell non-Hodgkin's lymphoma (NHL). Rituximab has since become a mainstay of treatment for low-grade NHL and over 400,000 patients worldwide have been treated with rituximab. Despite this extensive clinical experience, the mechanism of action of rituximab remains unclear, as does the nature of resistance.

Rituximab is a chimeric antibody consisting of a murine CD20-binding variable region linked to human IgG1 constant region. CD20 is a cell surface protein expressed on B-lymphocytes. CD20 has four transmembrane domains and has been proposed to act as an ion channel; however, the function of CD20 remains poorly understood.

Phase II trials of rituximab in people with refractory or relapsed low grade or follicular NHL demonstrated a response rate of approximately 50%. While the nature of de novo resistance to rituximab is unclear, such resistance is very rarely due to loss of the CD20 antigen, which cannot be shed or internalized and is rarely down-regulated. Despite these properties of CD20, acquired resistance to rituximab is common in that only half of patients previously responding to rituximab will respond to a second course of treatment.

To date, there is no effective way known to the inventors to predict whether or not a patient will respond to rituximab therapy. A diagnostic protocol which could provide information as to whether a patient is or is not responsive to rituximab would be desirable for a number of reasons, including avoidance of delays in alternative treatments, elimination of exposure to adverse effects of rituximab and reduction of unnecessary expense.

As such, there is interest in the development of a protocol that can accurately predict whether or not a patient is responsive to rituximab therapy.

### SUMMARY OF THE INVENTION

Methods are provided for determining whether a subject suffering from a neoplastic condition, e.g., non-Hodgkin's lymphoma (NHL), such as follicular lymphoma, is responsive to a particular therapy, such as antibody therapy, (e.g., rituximab therapy). In practicing the subject methods, an expression profile is obtained from the subject suffering from the neoplastic condition and employed to determine whether the subject is responsive to the therapy, e.g., antibody therapy, of interest. In addition, reagents and kits thereof that find use in practicing the subject methods are provided.

### BRIEF DESCRIPTION OF THE FIGURES

FIG. 1. Patterns of gene expression patterns in follicular lymphoma (FL) lymph nodes from rituximab responders cluster with normal lymphoid tissue. 20 FL lymph node samples from 16 patients and four normal lymphoid tissues (two tonsil and two spleen) from four different patients were sorted by hierarchical clustering based in similarity of gene expression. The resulting dendrogram is shown. Patient pathological diagnosis or normal tissue type, and response to rituximab treatment are shown. Samples are color coded by response to rituximab (for FL samples) or normal tissue for simplicity. For four patients, paired samples are presented; biopsy samples obtained later are identified by patient number plus “0.2”. In three cases (patients 6, 8, and 18), paired samples clustered together (black bars). The two samples from patient 17 clustered on separate branches of the dendrogram (arrows).

FIG. 2. Expression of genes identified as significantly different between rituximab responders and non-responders. Wilcoxon rank sum test was used to generate a list of 111 cDNAs with significantly increased expression (p≦0.004) in lymph node tissue from rituximab responders versus non-responders. Expression data for these genes is displayed as a hierarchical cluster the expression pattern of these genes in FL samples from 24 patients. A row in the cluster indicates expression of a specific gene across all 24 lymph node tissues. Gene names are listed to the right of each row. A column indicates the tissue in which the gene is expressed. The dendrogram is color coded based on outcome of rituximab treatment. Red, green, and black squares indicate that expression of the gene is greater than, less than, or equal to the median level of expression across all 24 lymph node tissues, respectively. Gray represents missing or poor quality data. The scale bar reflects the fold increase (red) or decrease (green) for any given gene relative to the median level of expression across all samples; numbers above the bar represent the log(2) scale values while the numbers below represent linear scale.

FIG. 3. A subset of genes with significantly higher expression in rituximab non-responders are listed by known biological function. A review of the literature for all genes in FIG. 11 identified 25 genes whose products have been demonstrated to function in cytokine, tumor necrosis factor, or T cell receptor signaling, or complement function, as indicated above.

FIG. 4 provides Table 1, which table lists genes found to have significantly higher expression in non-responders as measured on Lymphochip (LC) arrays and determine by Significance Analysis for Microarrays (SAM). 170 clones are identified with a median false discovery rate of 23.7%.

FIG. 5 provides Table 2, which table lists genes found to have significantly higher expression in non-responders as measured on SH arrays and determine by Significance Analysis for Microarrays (SAM). 144 Clones are identified with a median false discovery rate of 5.5%.

FIG. 6 provides Table 3, which table lists genes displaying higher mRNA levels in rituximab non-responders as identified by Wilcoxon rank sum test with p<0.004.

FIG. 7 provides Table 4, which table lists genes displaying higher mRNA levels in rituximab responders as identified by Wilcoxon rank sum test with p<0.004.)

FIG. 8 provides Table 5, which table lists 147 Genes significantly elevated in rituximab non-responders as determined by SAM.

FIG. 9 provides a list of the genes that display significantly increased expression in follicular lymphoma patients who responded to rituximab. Clone identification number, gene symbol and Unigene gene cluster are displayed. P-values were determined by Wilcoxon rank-sum test. SH=Stanford Human array. LC=Lymphochip array.

FIG. 10 provides a list of the genes that display significantly increased expression in follicular lymphoma patients who did not respond to rituximab. Clone identification number, gene symbol and Unigene gene cluster are displayed. P-values were determined by Wilcoxon rank-sum test.

FIGS. 11 and 12 are tables produced by combining the SH and LC datasets. Genes are only listed if they have a P value less than 0.005 in one set and 0.05 in the other for genes measured on both array types. If the clone/gene was present on only one array type, it is included if the P-value is less than 0.005 and N.M. is used to indicate that it was not present on the other array. FIG. 11 with FIG. 10 lists genes increased in nonresponders and FIG. 12 with FIG. 9 lists genes increased in responders.

FIG. 13 provides a list of 139 genes that appear in the list of 191 genes published in Nau et al., Proc. Nat'l Acad. Sci. USA (2002) 99:1503-1508 and that were present on the lymphochip microarray.

FIG. 14 is a hierarchical cluster of 24 patient samples based on the expression pattern of the 139 genes appearing in FIG. 13. Hierarchical clustering of follicular lymphoma samples based on the expression of these genes separates responders and non-responders (P=0.012 by Fisher's Exact test). Accordingly, there is a biological basis for cell-mediated immunity in the separation of responders and non-responders.

DESCRIPTION OF THE SPECIFIC EMBODIMENTS Methods are provided for determining whether a subject suffering from a neoplastic condition, e.g., non-Hodgkin's lymphoma (NHL), such as follicular lymphoma, is responsive to a particular therapy, such as antibody therapy, (e.g., rituximab therapy). In practicing the subject methods, an expression profile is obtained from the subject suffering from the neoplastic condition and employed to determine whether the subject is responsive to the therapy, e.g., antibody therapy, of interest. In addition, reagents and kits thereof that find use in practicing the subject methods are provided.

Before the subject invention is described further, it is to be understood that the invention is not limited to the particular embodiments of the invention described below, as variations of the particular embodiments may be made and still fall within the scope of the appended claims. It is also to be understood that the terminology employed is for the purpose of describing particular embodiments, and is not intended to be limiting. Instead, the scope of the present invention will be established by the appended claims.

In this specification and the appended claims, the singular forms “a,” “an” and “the” include plural reference unless the context clearly dictates otherwise. Unless defined otherwise, all technical and scientific terms used herein have the same meaning as commonly understood to one of ordinary skill in the art to which this invention belongs.

Where a range of values is provided, it is understood that each intervening value, to the tenth of the unit of the lower limit unless the context clearly dictates otherwise, between the upper and lower limit of that range, and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, the preferred methods, devices and materials are now described.

All publications mentioned herein are incorporated herein by reference for the purpose of describing and disclosing the subject components of the invention that are described in the publications, which components might be used in connection with the presently described invention.

As summarized above, the subject invention is directed to methods of determining whether a subject suffering from a neoplastic condition is responsive to a particular therapy, such as antibody therapy, as well as reagents and kits for use in practicing the subject methods. In further describing the invention, the subject methods are described first, followed by a review of the reagents and kits for use in practicing the subject methods.

Methods of Determining Whether a Subject Suffering from a Neoplastic Condition is Responsive to a Particular Therapy

The subject invention provides methods of determining whether a patient or subject suffering from a neoplastic disease, i.e., hyperproliferative disorder, is responsive to a particular therapy, such as antibody therapy. Hyperproliferative disorders, or malignancies, are conditions in which there is unregulated cell growth. The methods of the present invention are directed at hyperproliferative disorders and particularly whether such a disorder will or will not be responsive to a particular antineoplastic therapy, e.g., antibody therapy. The disorder may be characterized by the presence or absence of solid tumors.

In certain embodiments, the subject methods are directed to determining whether a B-cell hyperproliferative disorder, e.g., NHL, is responsive to therapeutic antibody therapy. B-cell hyperproliferative disorders are those disorders that derive from cells in the B cell lineage, typically including hematopoietic progenitor cells expressing B lineage markers, pro-B cells, pre-B cells, B-cells and memory B cells; and that express markers typically found on such B lineage cells.

Of particular interest are non-Hodgkin's lymphomas (NHLs), which are a heterogeneous group of lymphoproliferative malignancies with differing patterns of behavior and responses to treatment. Like Hodgkin's disease, NHL usually originates in lymphoid tissues and can spread to other organs, however, NHL is much less predictable than Hodgkin's disease and has a far greater predilection to disseminate to extranodal sites. The NHLs can be divided into 2 prognostic groups: the indolent lymphomas and the aggressive lymphomas. Indolent NHL types have a relatively good prognosis, with median survival in the range of 10 years, but they usually are not curable in advanced clinical stages. The aggressive type of NHL has a shorter natural history. A number of these patients can be cured with intensive combination chemotherapy regimens, but there is a significant number of relapses, particularly in the first 2 years after therapy.

Among the NHL are a variety of B-cell neoplasms, including precursor B-lymphoblastic leukemia/lymphoma; peripheral B-cell neoplasms, e.g. B-cell chronic lymphocytic leukemia; prolymphocytic leukemia; small lymphocytic lymphoma; mantle cell lymphoma; follicle center cell lymphoma; marginal zone B-cell lymphoma; splenic marginal zone lymphoma; hairy cell leukemia; diffuse large B-cell lymphoma; T-cell rich B-cell lymphoma, Burkitt's lymphoma; high-grade B-cell lymphoma, (Burkitt-like); etc.

Follicular lymphoma comprises 70% of the indolent lymphomas reported in American and European clinical trials. Most patients with follicular lymphoma are over age 50 and present with widespread disease at diagnosis. Nodal involvement is most common, often accompanied by splenic and bone marrow disease. The vast majority of patients with advanced stage follicular lymphoma are not cured with current therapeutic options, and the rate of relapse is fairly consistent over time, even in patients who have achieved complete responses to treatment. Subtypes include follicular small cleaved cell (grade 1) and follicular mixed small cleaved and large cell (grade 2). Another subtype of interest is follicular large cell (grade 3 or FLC) lymphoma which can be divided into grades 3a and 3b.

Marginal zone lymphomas were previously included among the diffuse small lymphocytic lymphomas. When marginal zone lymphomas involve the nodes, they are called monocytoid B-cell lymphomas, and when they involve extranodal sites (gastrointestinal tract, thyroid, lung, breast, skin), they are called mucosa-associated lymphatic tissue (MALT) lymphomas. Many patients have a history of autoimmune disease, such as Hashimoto's thyroiditis or Sjogren's syndrome, or of Helicobacter gastritis. Most patients present with stage I or II extranodal disease, which is most often in the stomach. When disseminated to lymph nodes, bone marrow, or blood, this entity behaves like other low-grade lymphomas. Large B-cell lymphomas of MALT sites are classified and treated as diffuse large cell lymphomas.

Splenic marginal zone lymphoma is an indolent lymphoma that is marked by massive splenomegaly and peripheral blood and bone marrow involvement, usually without adenopathy. This type of lymphoma is otherwise known as splenic lymphoma with villous lymphocytes, an uncommon variant of B-cell chronic lymphocytic leukemia. Management of this entity usually starts with splenectomy which is different than other low-grade lymphomas. If/when the disease progresses after splenectomy, it tends to be managed like other low grade lymphomas.

Among the aggressive forms of NHL is diffuse large B-cell lymphoma, which is the most common of the non-Hodgkin's lymphomas, comprising 30% of newly-diagnosed cases. Most patients present with rapidly enlarging masses, often with symptoms both locally and systemically. Relapses after treatment are not uncommon, depending on the presence of various risk factors. Lymphomatoid granulomatosis is an EBV positive large B-cell lymphoma with a predominant T-cell background. The histology shows association with angioinvasion and vasculitis, usually manifesting as pulmonary lesions or paranasal sinus involvement. Patients are managed like others with diffuse large cell lymphoma.

Primary mediastinal B-cell lymphoma is a subset of diffuse large cell lymphoma characterized by significant fibrosis on histology. Patients are usually female and young. Patients present with a locally invasive anterior mediastinal mass which may cause respiratory symptoms or superior vena cava syndrome. Therapy and prognosis are the same as for other comparably-staged patients with diffuse large cell lymphoma, except for advanced-stage patients with a pleural effusion, who have an extremely poor prognosis (progression-free survival is less than 20%) whether the effusion is cytologically positive or negative.

Mantle cell lymphoma is found in lymph nodes, the spleen, bone marrow, blood, and sometimes the gastrointestinal system (lymphomatous polyposis). Mantle cell lymphoma is characterized by CD5-positive follicular mantle B cells, a translocation of chromosomes 11 and 14, and an overexpression of the cyclin D1 protein. The median survival is significantly shorter (3-5 years) than that of other lymphomas, and this histology is now considered to be an aggressive lymphoma. A diffuse pattern and the blastoid variant have an aggressive course with shorter survival, while the mantle zone type may have a more indolent course. Refractoriness to chemotherapy is a usual feature.

Lymphoblastic lymphoma is a very aggressive form of NHL. It often occurs in young patients, but not exclusively. It is commonly associated with large mediastinal masses and has a high predilection for disseminating to bone marrow and the central nervous system (CNS). Treatment is usually patterned after that for acute lymphoblastic leukemia (ALL). Since these forms of NHL tend to progress so quickly, combination chemotherapy is instituted rapidly once the diagnosis has been confirmed. Careful review of the pathologic specimens, bone marrow aspirate and biopsy specimen, cerebrospinal fluid cytology, and lymphocyte marker constitute the most important aspects of the pretreatment staging work-up.

Burkift's lymphoma/diffuse small noncleaved cell lymphoma typically involves younger patients and represents the most common type of pediatric non-Hodgkin's lymphoma. These aggressive extranodal B-cell lymphomas are characterized by translocation and deregulation of the c-myc gene on chromosome 8. A subgroup of patients with dual translocation of c-myc and bcl-2 appear to have an extremely poor outcome despite aggressive therapy. Treatment of Burkitt's lymphoma/diffuse small noncleaved cell lymphoma involves aggressive multidrug regimens similar to those used for the advanced-stage aggressive lymphomas.

Patients who undergo transplantation of the heart, lung, liver, kidney, or pancreas usually require life-long immunosuppression. Life-long immunosuppression may result in post-transplantation lymphoproliferative disorder, which appears as an aggressive lymphoma. Pathologists can distinguish a polyclonal B-cell hyperplasia from a monoclonal B-cell lymphoma; both are almost always associated with EBV. In some cases, usually for the polyclonal forms of the disease, withdrawal of immunosuppression results in eradication of the lymphoma. When this is unsuccessful or not feasible, a combination therapy is used. EBV-negative post-transplantation lymphoproliferative disorders occur late and have a particularly poor prognosis. Chronic lymphocytic leukemia (CLL) is a disorder of morphologically mature but immunologically less mature lymphocytes and is manifested by progressive accumulation of these cells in the blood, bone marrow, and lymphatic tissues. Lymphocyte counts in the blood are usually equal to or higher than 10,000 per cubic millimeter. At present there is no curative therapy. CLL occurs primarily in middle-aged and elderly individuals, with increasing frequency in successive decades of life. The clinical course of this disease progresses from an indolent lymphocytosis without other evident disease to one of generalized lymphatic enlargement with concomitant pancytopenia. Complications of pancytopenia, including hemorrhage and infection, represent a major cause of death in these patients. Immunological aberrations, including Coombs-positive hemolytic anemia, immune thrombocytopenia, and depressed immunoglobulin levels may all complicate the management of CLL. CLL lymphocytes coexpress the B-cell antigens CD19 and CD20 along with the T-cell antigen CD5. CLL B cells express relatively low levels of surface-membrane immunoglobulin (compared with normal peripheral blood B cells) and a single light chain (kappa or lambda). CLL is diagnosed by an absolute increase in lymphocytosis and/or bone marrow infiltration coupled with the characteristic features of morphology and immunophenotype.

AIDS-related lymphomas are comprised of a narrow spectrum of histologic types consisting almost exclusively of B-cell tumors of aggressive type. These include diffuse large cell lymphoma; B-immunoblastic; and small non-cleaved, either Burkitt's or Burkitt's like. The HIV-associated lymphomas can be categorized into: primary central nervous system lymphoma (PCNSL); systemic lymphoma; and primary effusion lymphoma. All of these lymphomas differ from non-HIV-related lymphomas in their molecular characteristics, presumed mechanism of pathogenesis, treatment, and clinical outcome. All 3 pathologic types are equally distributed and represent aggressive disease. In general, the clinical setting and response to treatment of patients with AIDS-related lymphoma is very different from the non-HIV patients with lymphoma. The HIV-infected individual with aggressive lymphoma usually presents with advanced-stage disease that is frequently extranodal. The clinical course is more aggressive, and the disease is both more extensive and less responsive to chemotherapy. Immunodeficiency and cytopenias, common in these patients at the time of initial presentation, are exacerbated by the administration of chemotherapy. Therefore, treatment of the malignancy increases the risk of opportunistic infections that, in turn, further compromise the delivery of adequate treatment.

Acute lymphocytic leukemia (ALL) generally has an aggressive course, depending in part on the presence of the Philadelphia (Ph) chromosome. Patients with Ph chromosome-positive ALL are rarely cured with chemotherapy. Many patients who have molecular evidence of the bcr-abl fusion gene, which characterizes the Ph chromosome, have no evidence of the abnormal chromosome by cytogenetics.

Although the methods of the invention are primarily applied to NHL, in some cases treatment may be used in cases of Hodgkin's lymphoma, which is a lymphoma characterized by a pleomorphic lymphocytic infiltrate with malignant multinucleated giant cells. Most cases of Hodgkin's disease probably arise from germinal center B cells that are unable to synthesize immunoglobulin. The majority of cases in developing countries and about one third of those in the United States are associated with the presence of Epstein-Barr virus in the Reed-Sternberg cells. Treatment strategies depend on a number of factors including the presence of B symptoms, the histologic subtype, gender, and sexual maturity. To date there are several published studies demonstrating the effectiveness of Rituxan for CD20-positive Hodgkin's disease, particularly the lymphocyte predominant variant.

Other neoplastic disease conditions whose responsiveness to antibody therapy can be evaluated according to the subject methods include, but are not limited to: colorectal cancer, non-small cell lung cancer, small cell lung cancer, ovarian cancer, breast cancer, head and neck cancer, renal cell carcinoma, and the like.

As summarized above, the subject methods may be used to evaluate the responsive of a subject to a given antineoplastic therapy. Antineoplastic therapies of interest include, but are not limited to: chemotherapy, radiation therapy, antibody therapy, etc.

By therapeutic antibody therapy is meant a treatment protocol or regimen that includes administration of a therapeutic affinity or antibody agent. Representative therapeutic antibody agents specifically bind to antigens present on B cells, particularly hyperproliferative B cells, e.g. B lineage lymphomas and leukemias, and the like. The term “antibody” is used in the broadest sense and specifically covers monoclonal antibodies (including full length monoclonal antibodies), polyclonal antibodies, multispecific antibodies (e.g., bispecific antibodies), and antibody fragments so long as they exhibit the desired biological activity. Fragments comprise a portion of a full-length antibody, generally the antigen binding or variable region thereof. Examples of antibody fragments include Fab, Fab′, F(ab′)₂, and Fv fragments; diabodies; linear antibodies; single-chain antibody molecules; and multispecific antibodies formed from antibody fragments. In some aspects of the invention, a combination of one or more antibodies with different specificities, either for epitopes of a single antigen, or for multiple antigens, may be used.

Markers that are specifically found on B cells include B220 epitope (CD45R), which is an exon specific epitope found on essentially all B cells, and is maintained throughout B cell development (Coffman et al. (1982) Immunol. Rev. 69:5-23). The B cell markers CD19, CD20; CD22; CD23 are selectively expressed on B cells and have been associated with B cell malignancies (Kalil and Cheson (2000) Drugs Aging 16(1):9-27; U.S. Pat. No. 6,183,744, herein incorporated by reference). Surface immunoglobulin, including epitopes present on the constant regions or idiotypic determinants, is a specific marker for B cells and has been utilized in immunotherapy (Caspar et al. (1997) Blood 90(9):3699-706). The MB-1 antigen is found on all normal immunoglobulin (Ig)-expressing cells, but not on T cells, thymocytes, granulocytes, or platelets, and expressed by virtually all Ig-expressing B cell tumors (Link et al. (1986) J Immunol 137(9):3013-8). Other B cell antigens of interest known to be expressed, for example, on non-Hodgkin's lymphomas, are Muc-1; B5; BB1; and T9 (Freedman et al. (1987) Leukemia 1(1):9-15).

Of particular interest is the CD20 antigen, also known as “Bp35”. (Note that CD20 was called B1 early in the course of research on B-cell markers). CD20 is a human B cell marker that is expressed during early pre-B cell development and remains until plasma cell differentiation. The CD20 molecule may regulate a step in the activation process that is required for cell cycle initiation and differentiation, and is usually expressed at very high levels on neoplastic B cells. Thus, the CD20 surface antigen can be targeted for treating B cell lymphomas. U.S. Pat. No. 5,736,137, herein incorporated by reference, describes the chimeric antibody “C2B8” that binds the CD20 antigen and its use to treat B cell lymphoma (antibody is also known as Rituxan®, rituximab, Mabthera (this is a trademark in Europe)).

In a preferred embodiment, the antibody is a monoclonal antibody. Monoclonal antibodies are highly specific, being directed against a single antigenic site, and each monoclonal antibody is directed against a single determinant on the antigen. For example, the monoclonal antibodies to be used in accordance with the present invention may be made by the hybridoma method first described by Kohler et al. (1975) Nature 256:495, or may be made by recombinant DNA methods (see, e.g., U.S. Pat. No. 4,816,567). The monoclonal antibodies may also be isolated from phage antibody libraries using the techniques described in Clackson et al. (1991) Nature 352:624-628 (1991) and Marks et al. (1991) J. Mol. Biol. 222:581-597 (1991), for example. For clinical use, the monoclonal antibodies may be humanized forms of non-human antibodies. These are chimeric antibodies that contain sequences derived from both human and non-human immunoglobulin. For the most part, humanized antibodies are human immunoglobulins in which residues from a hypervariable region of the recipient are replaced by residues from a hypervariable region of a non-human species having the desired specificity, affinity, and capacity.

Specificity, as used herein, refers to the affinity of the antibody, and to the cross-reactivity with other antigens. In order to consider an antibody interaction to be “specific”, the affinity will be at least about 10⁻⁷ M, usually about 10^{-8 to-9} M, and may be up to 10⁻¹¹ or higher for the epitope of interest. It will be understood by those of skill in the art that the term “specificity” refers to such a high affinity binding, and is not intended to mean that the antibody cannot bind to other molecules as well. One may find cross-reactivity with different epitopes, due, e.g. to a relatedness of antigen sequence or structure, or to the structure of the antibody binding pocket itself. Antibodies demonstrating such cross-reactivity are still considered specific for the purposes of the present invention.

In practicing the subject methods, a subject or patient sample, e.g., cells or collections thereof, e.g., tissues, is assayed to determine whether the host from which the assayed sample was obtained is responsive to a given therapy, e.g., therapeutic antibody therapy. In practicing the subject diagnostic methods, the sample is assayed to obtain an expression profile for one or more genes, where the term expression profile is used broadly to include a genomic expression profile, e.g., an expression profile of mRNAs, or a proteomic expression profile, e.g., an expression profile of one or more different proteins.

In generating the expression profile, in many embodiments a sample is assayed to generate an expression profile that includes expression data for at least one gene/protein, usually a plurality of genes/proteins, where by plurality is meant at least two different genes/proteins, and often at least about 5, typically at least about 10 and more usually at least about 20 different genes/proteins or more, such as 50 or more, 100 or more, etc.

Genes/proteins of interest are genes/proteins that are differentially expressed or present at different levels in responders and non-responders for the particular disease condition and therapy therefor of interest. In certain embodiments, genes that are involved in cell mediated immunity, i.e., genes whose expression or lack thereof modulates or has an impact on cell mediated immunity (such as genes involved in macrophage activation). A representative list of genes involved in cell mediated immunity includes those genes listed in Table 14, as well as those genes appearing in Table 13 of FIG. 16. In such embodiments, one can determine a cell mediated immunity profile for a subject, e.g., an expression pattern of genes associated with cell mediated immunity or some other measure of a subject's cell mediate immunity profile, and then compare this profile to a control (as defined below) to make a determination about the antineoplastic therapy responsiveness of the subject. In such embodiments, the method performed is a method of identifying or determining a cell mediated immunity profile or signature (or other parameter) for a subject, and then comparing this profile to a control to determine whether the subject will or will not be responsive to the antineoplastic therapy of interest.

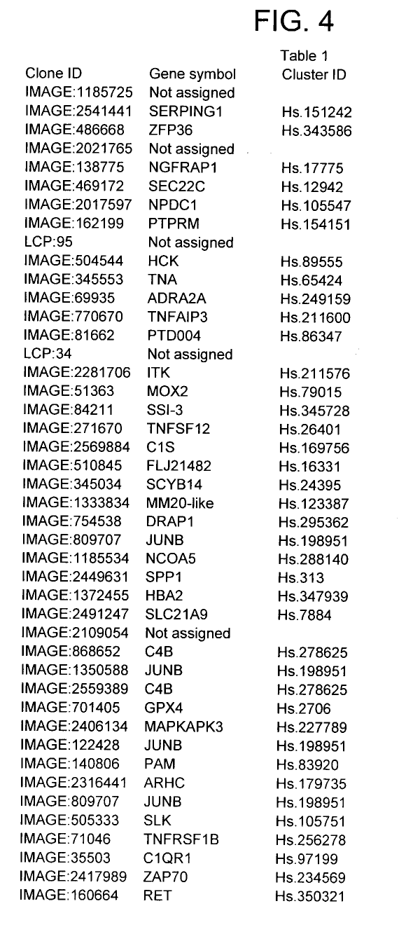

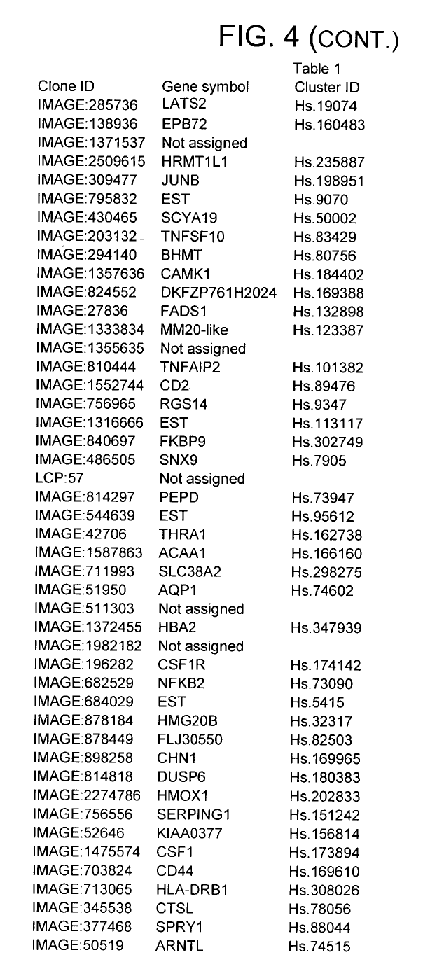

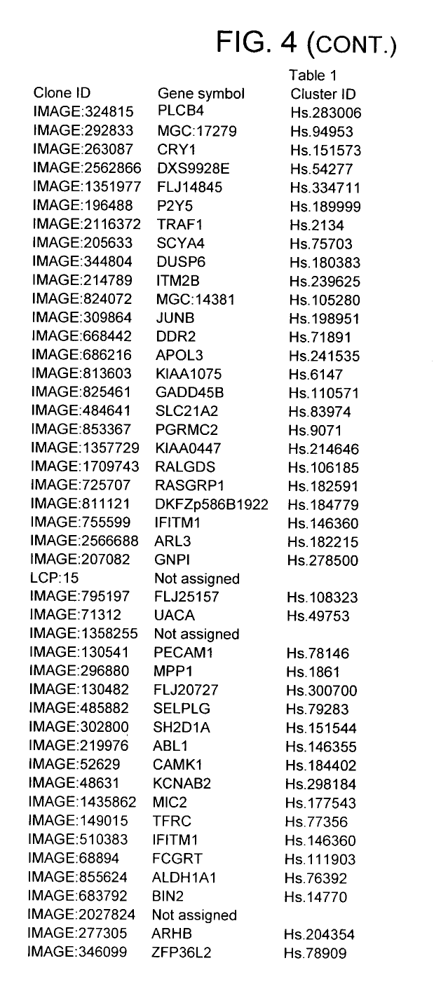

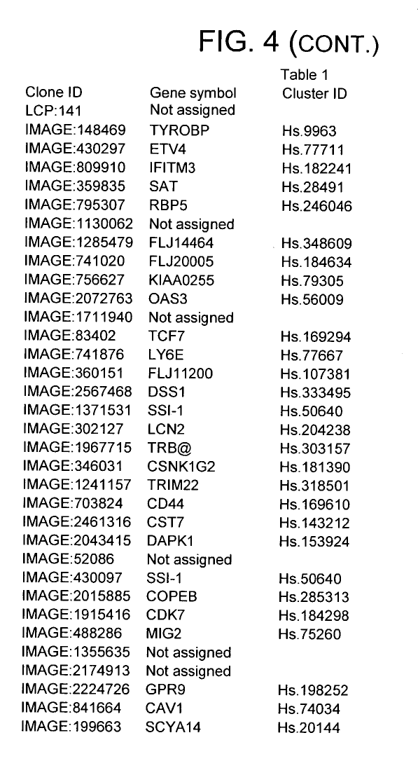

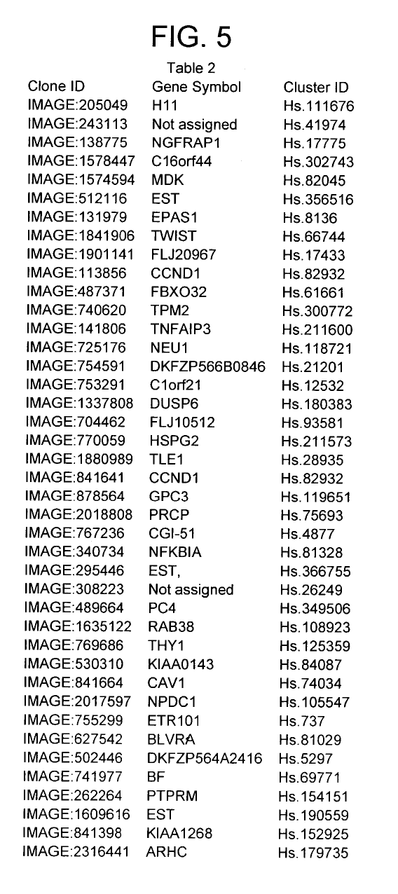

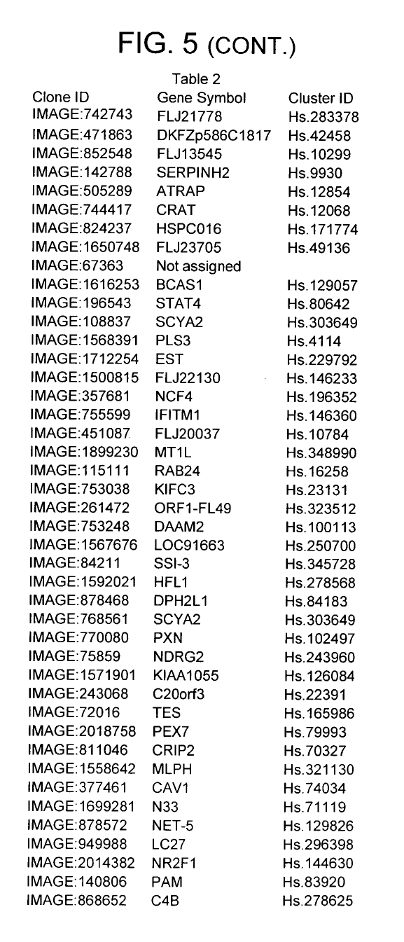

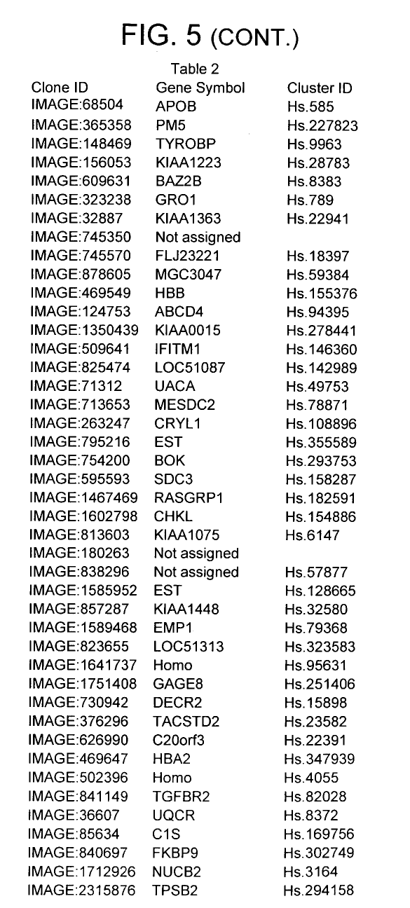

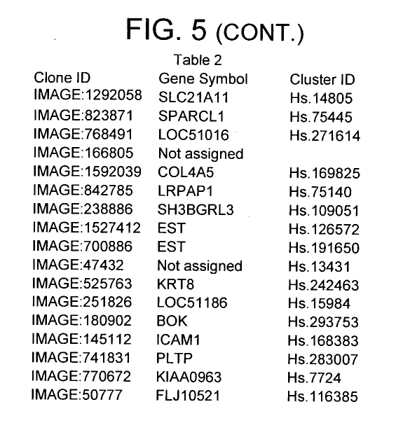

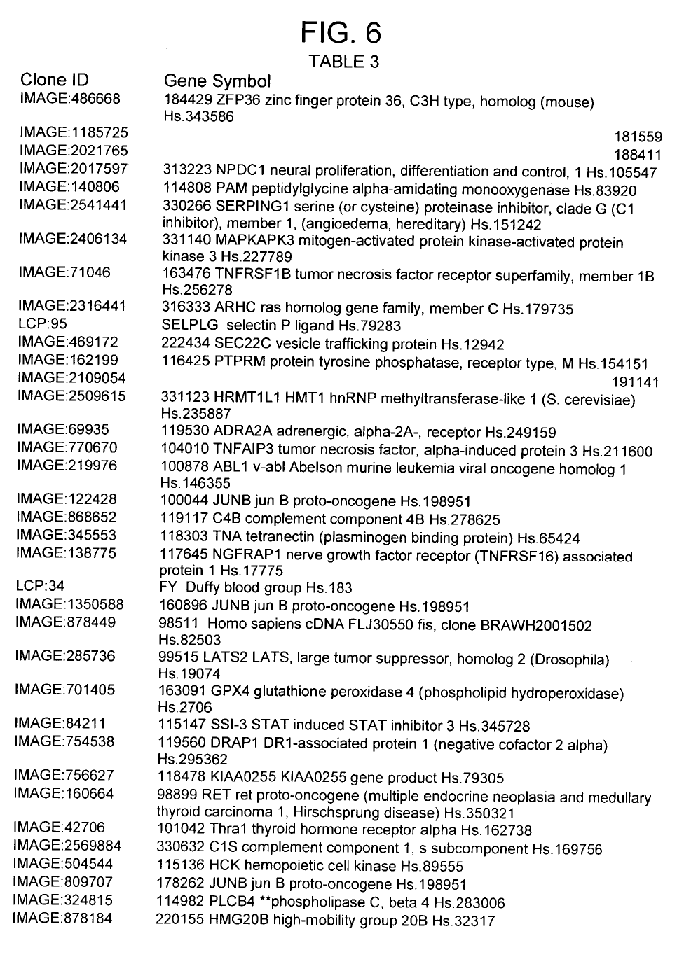

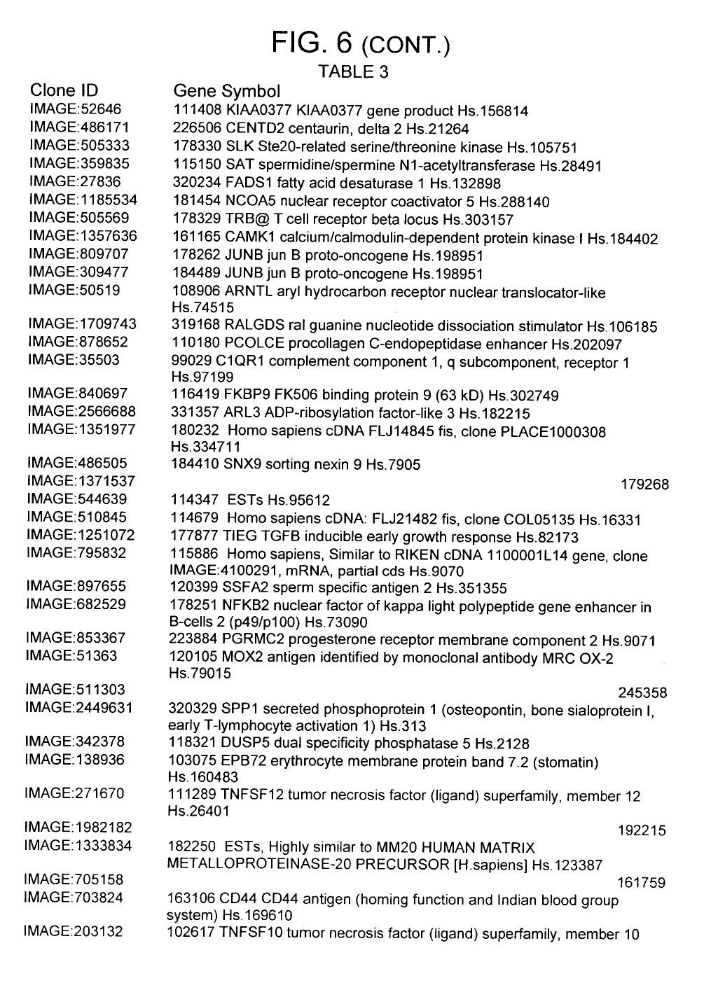

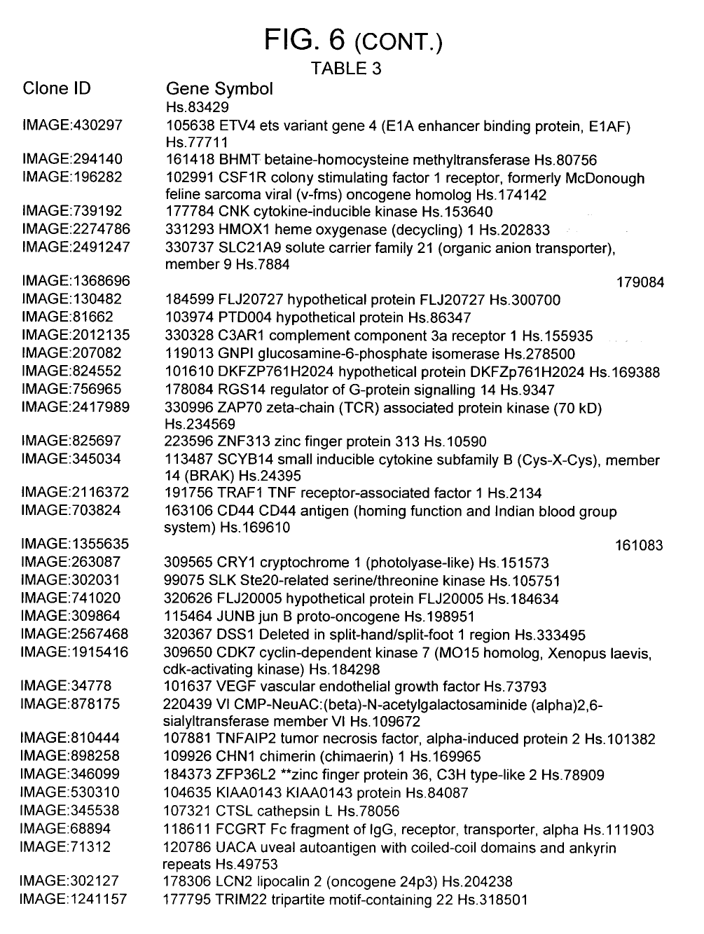

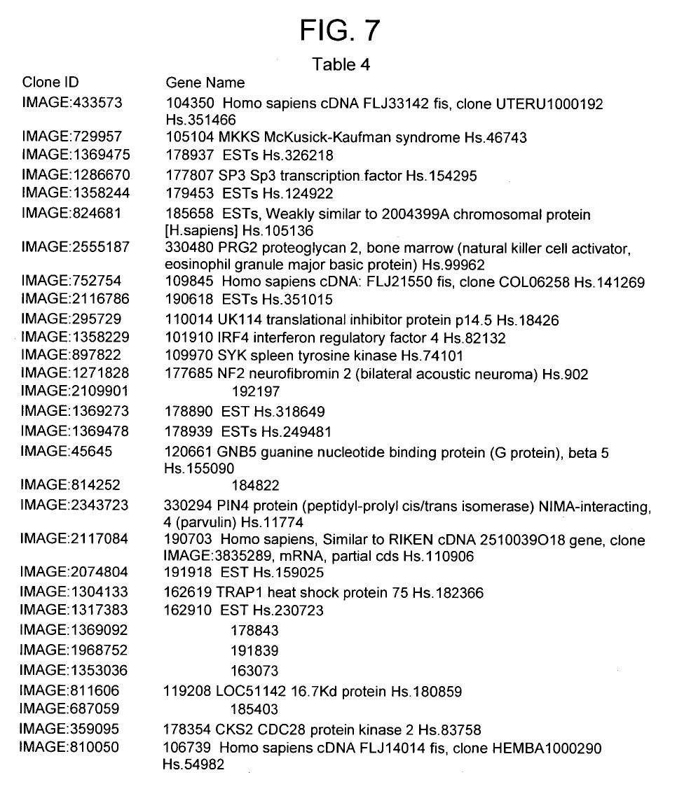

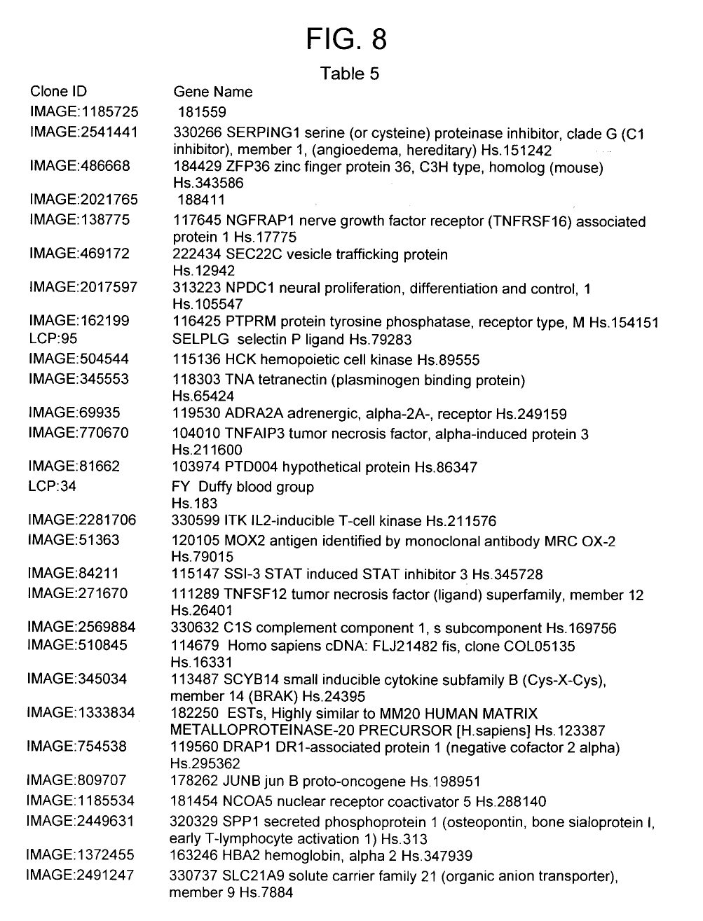

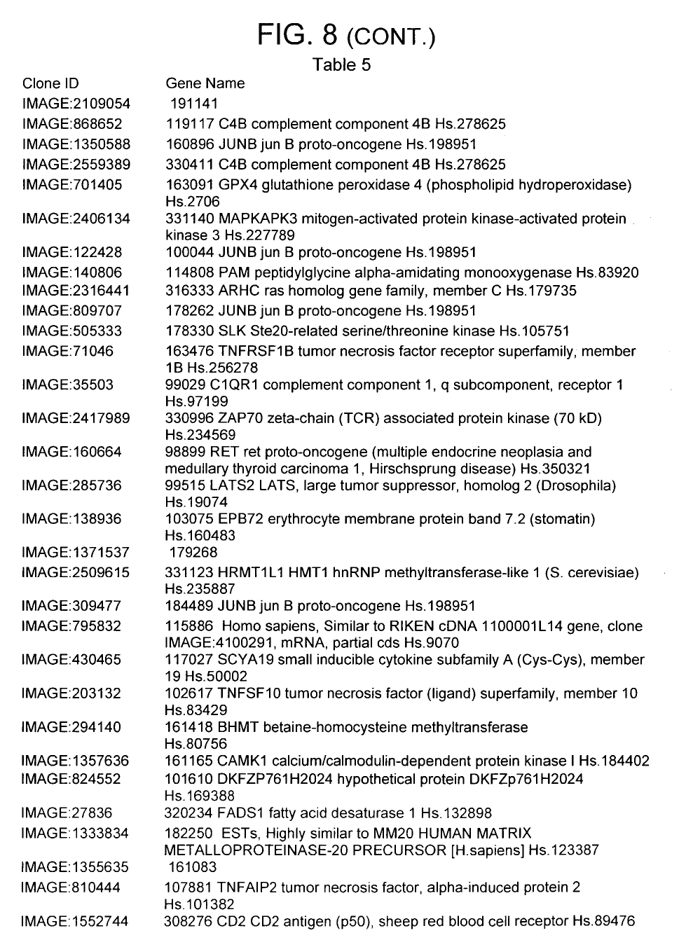

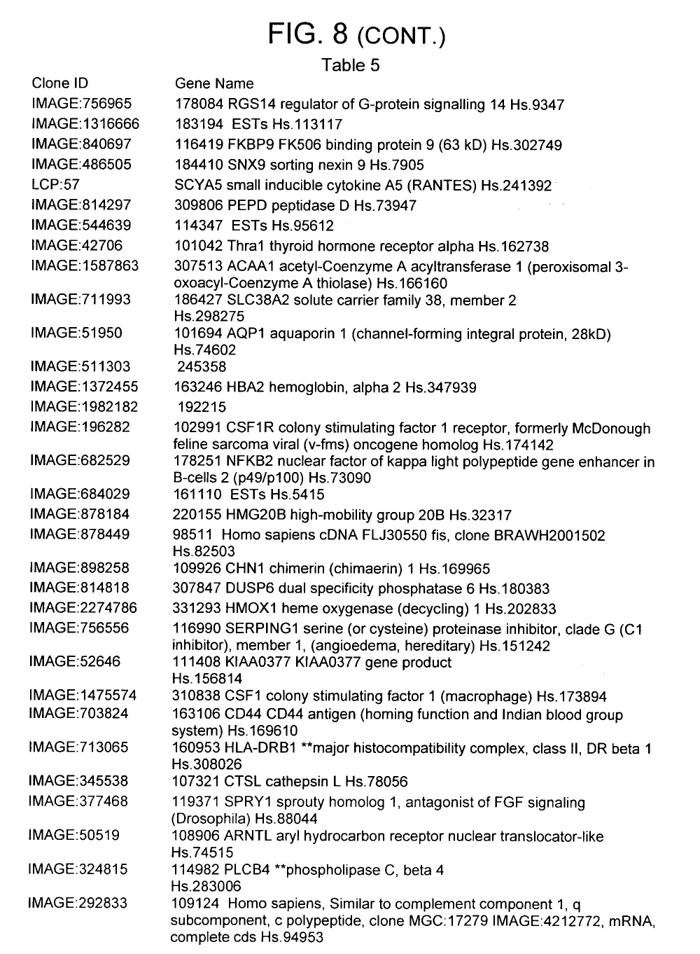

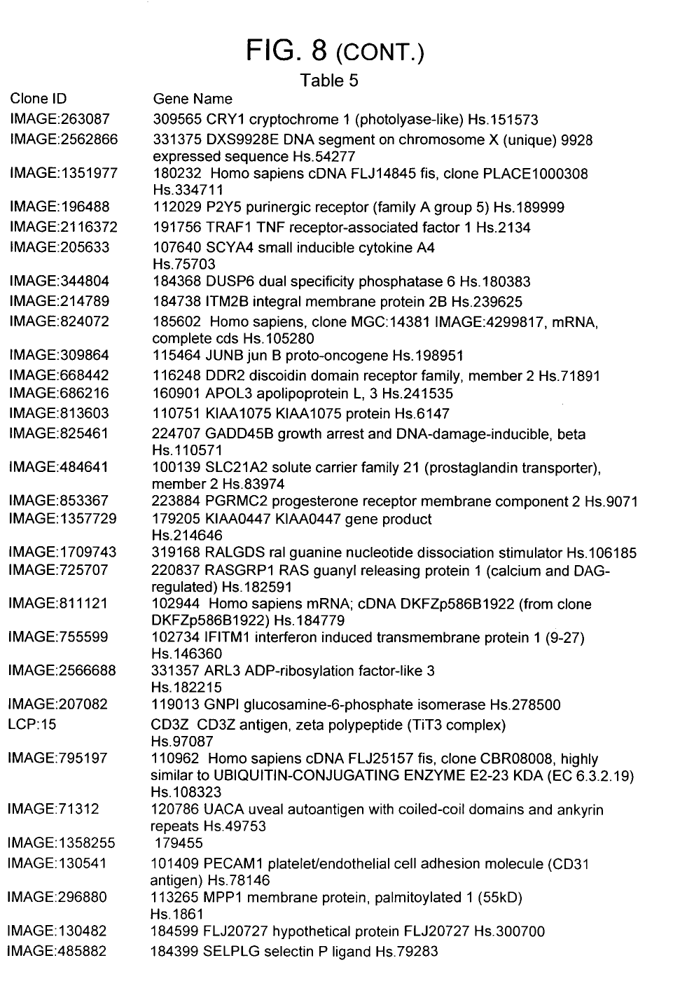

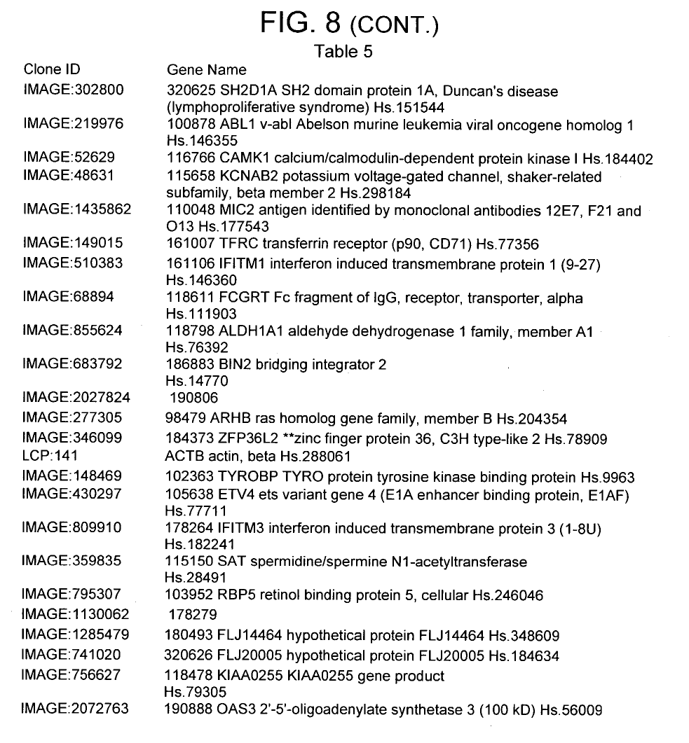

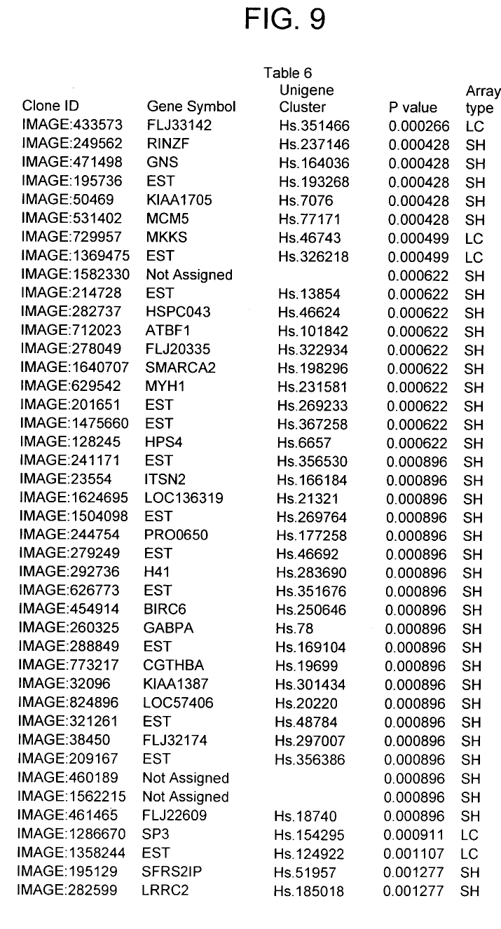

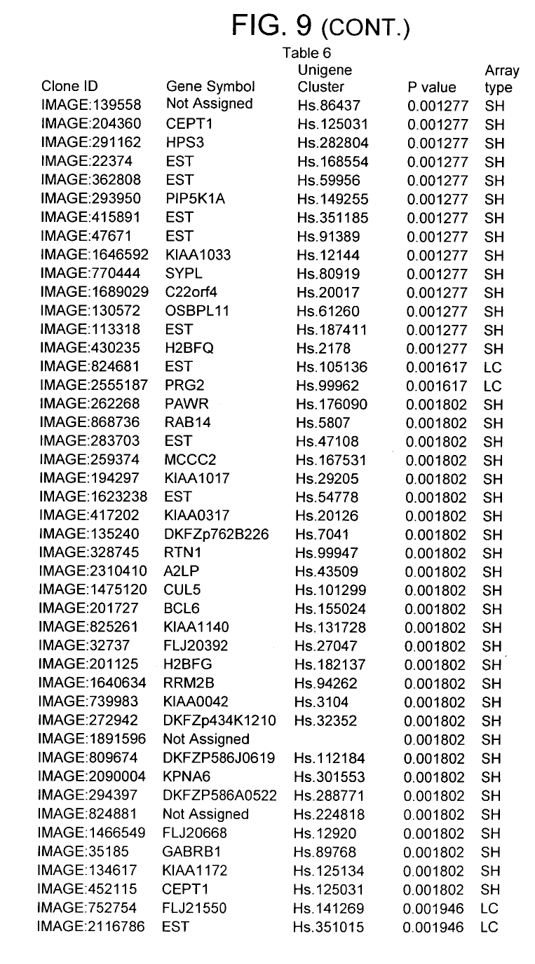

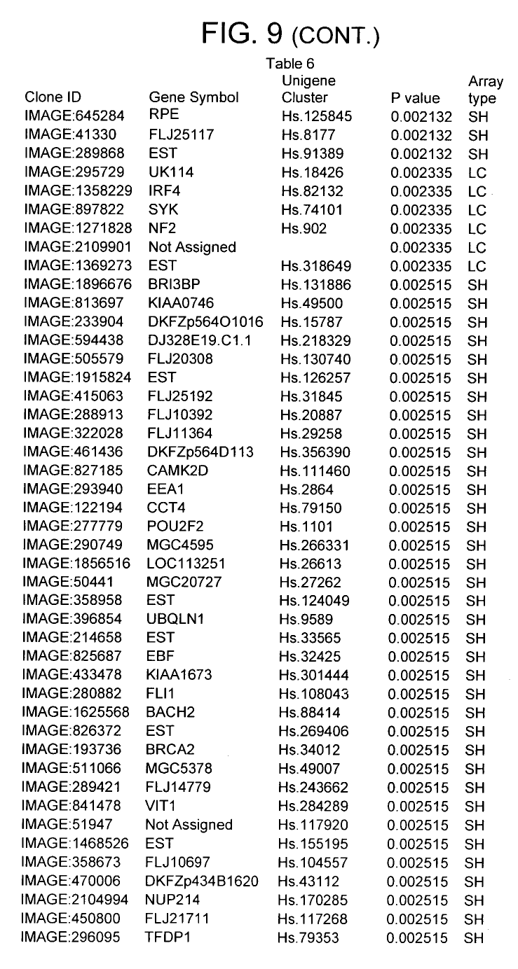

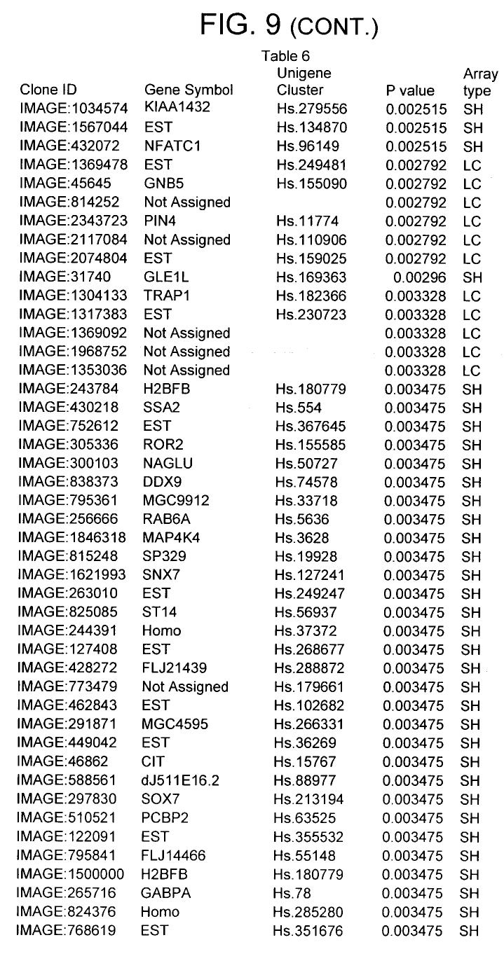

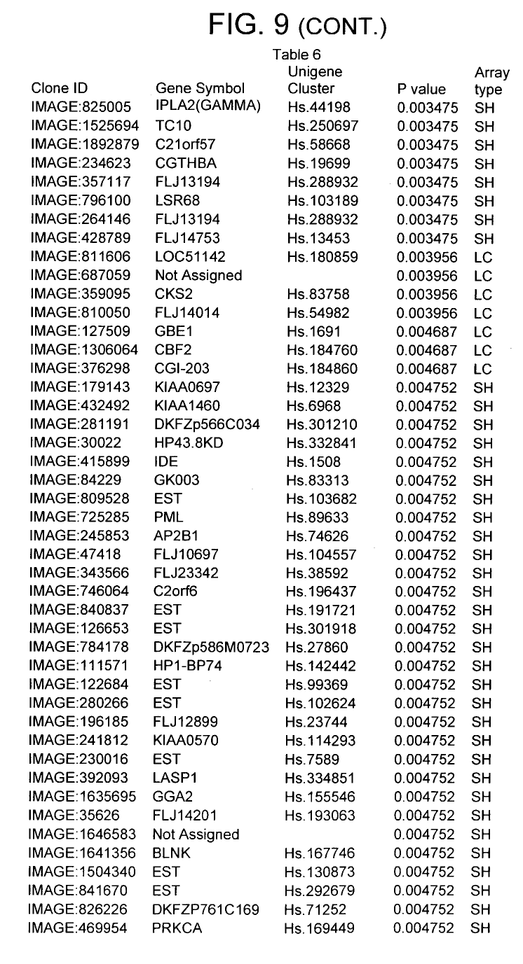

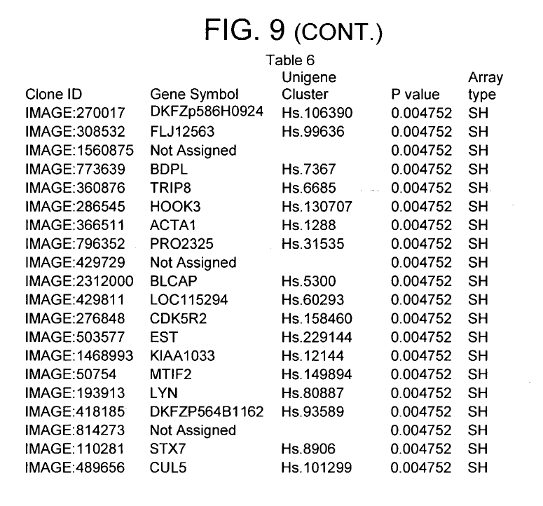

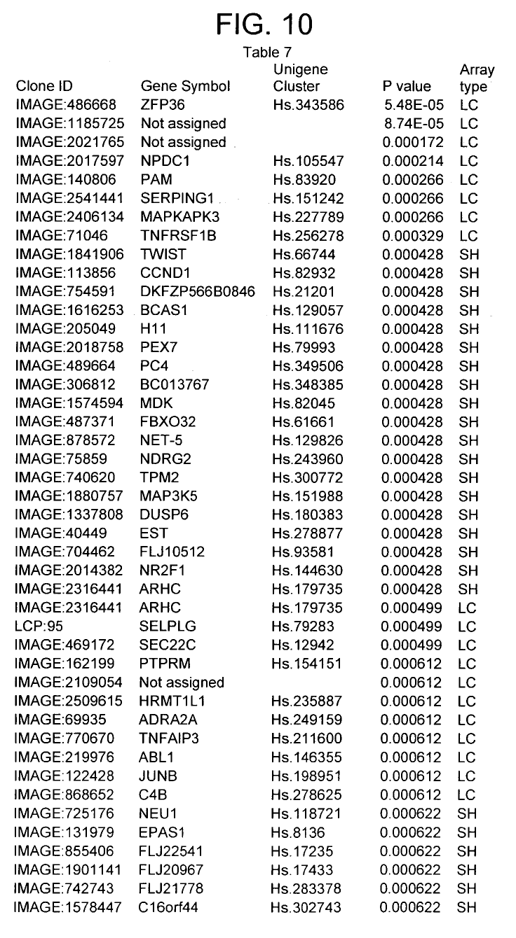

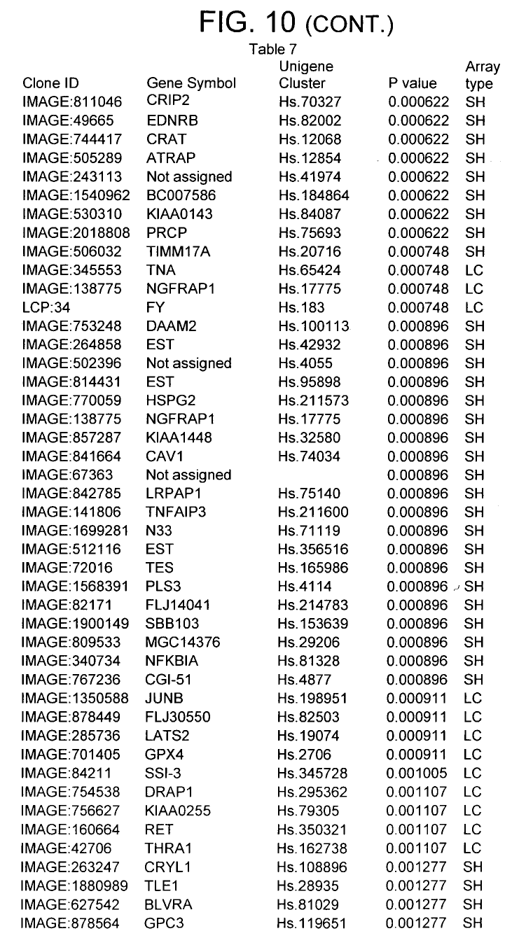

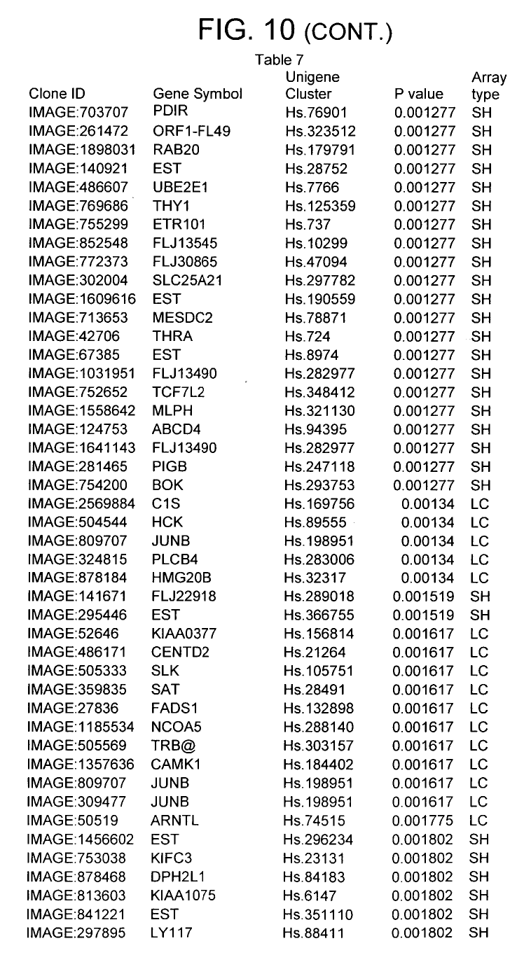

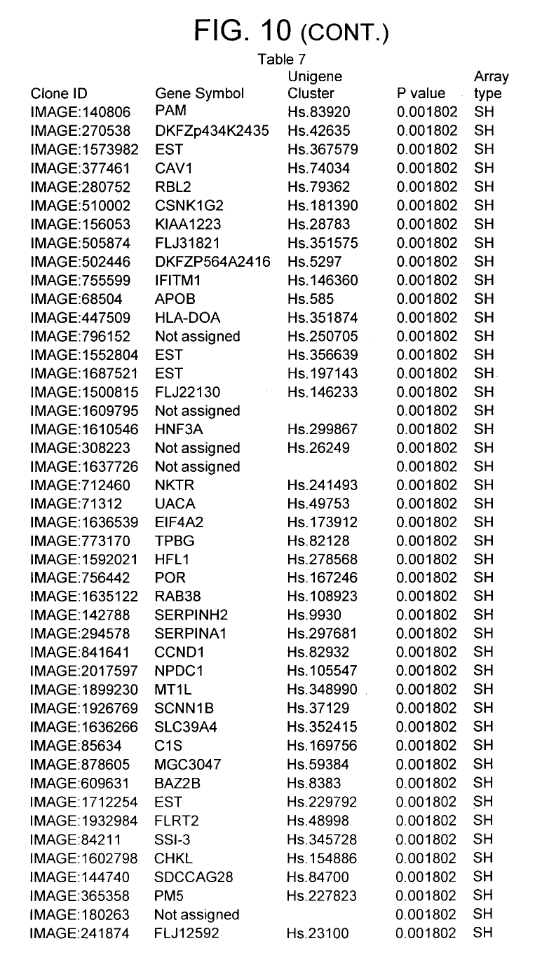

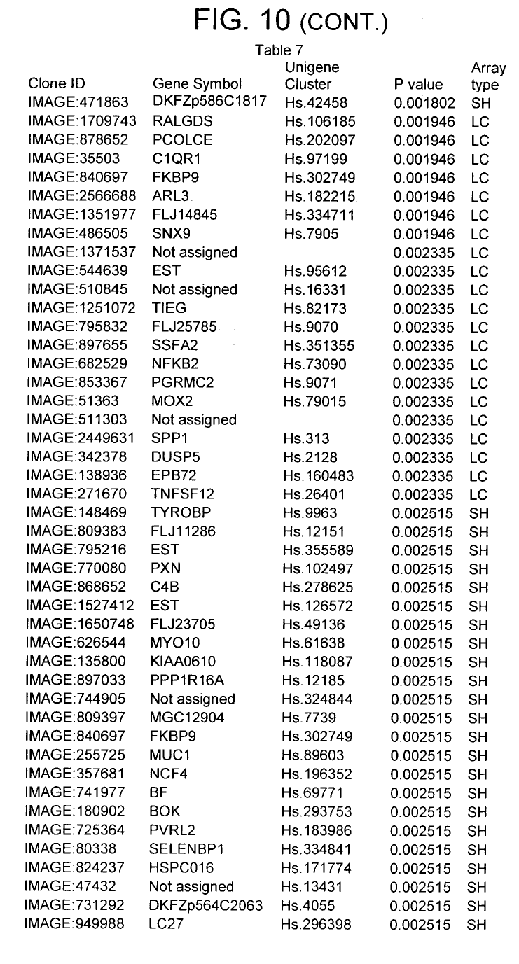

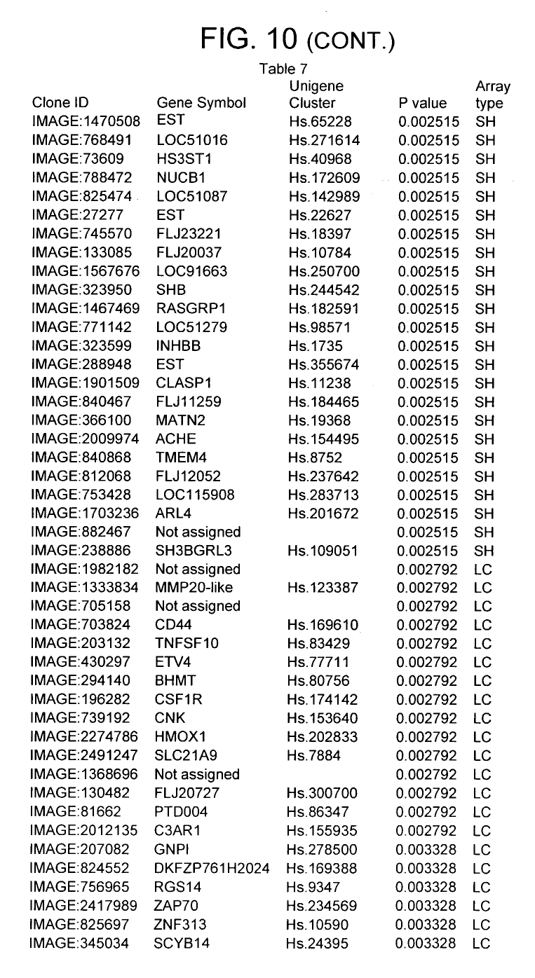

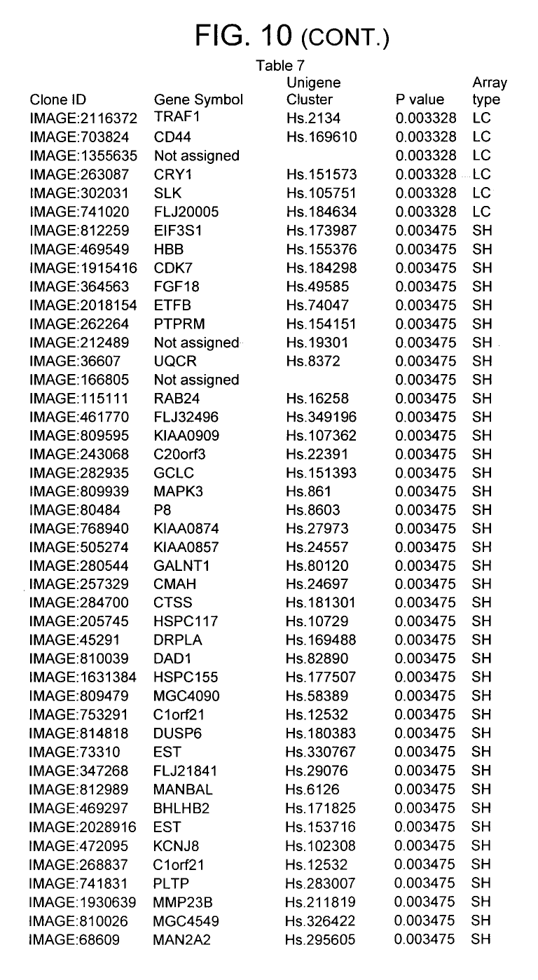

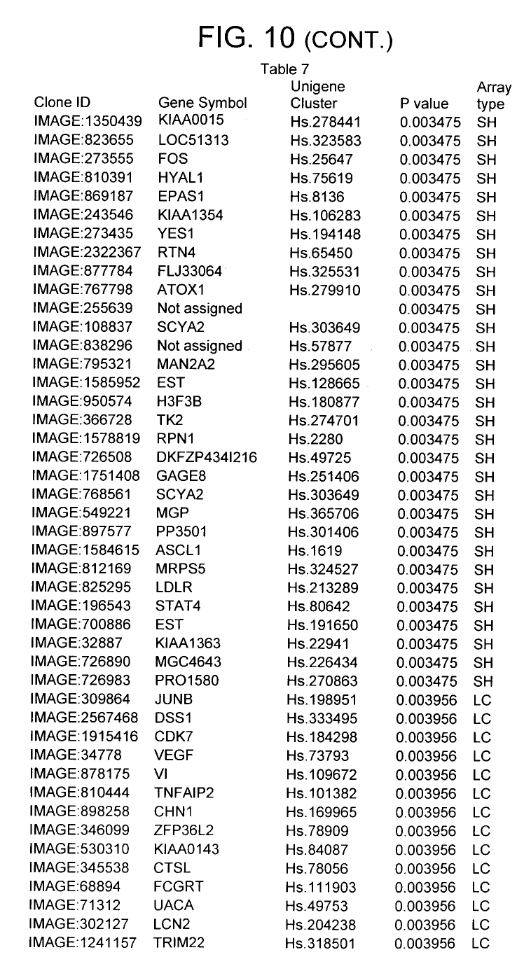

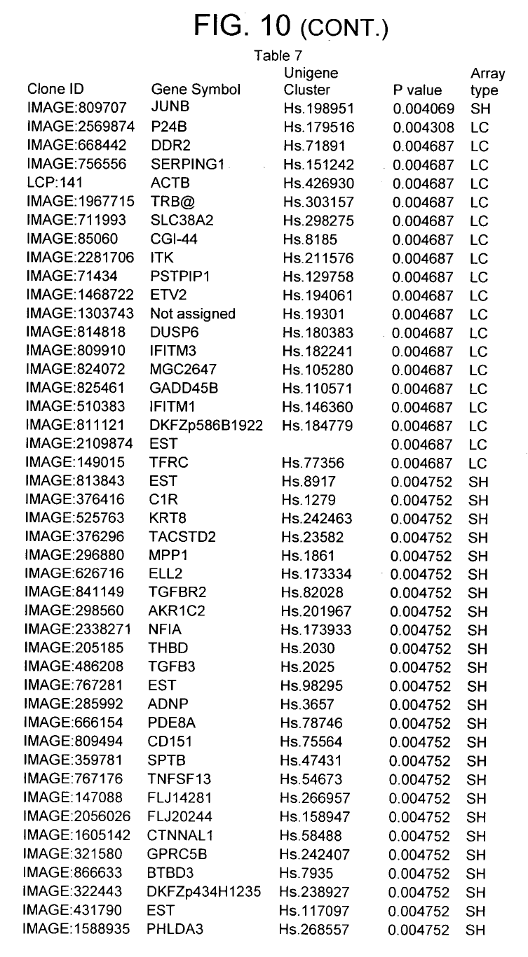

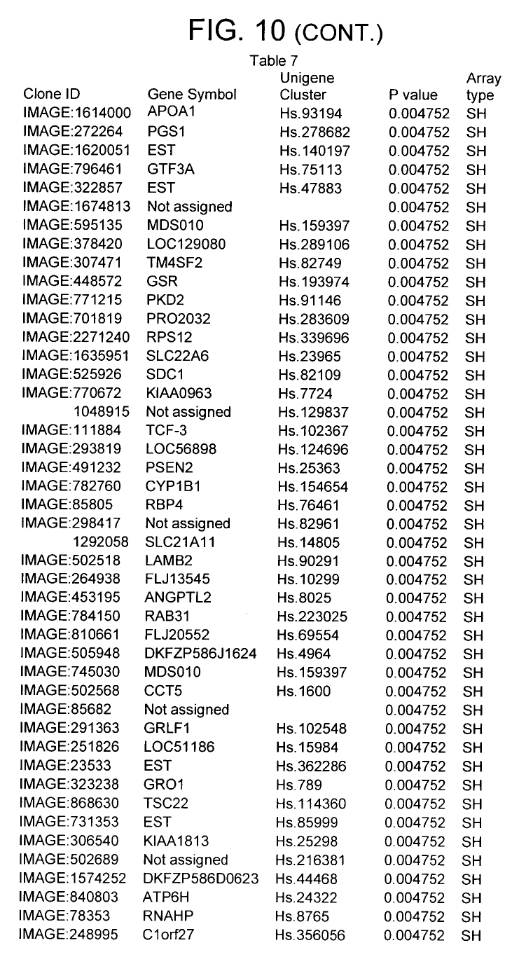

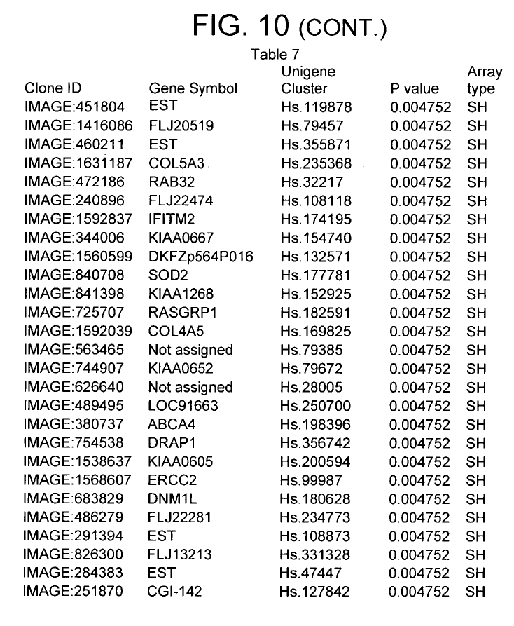

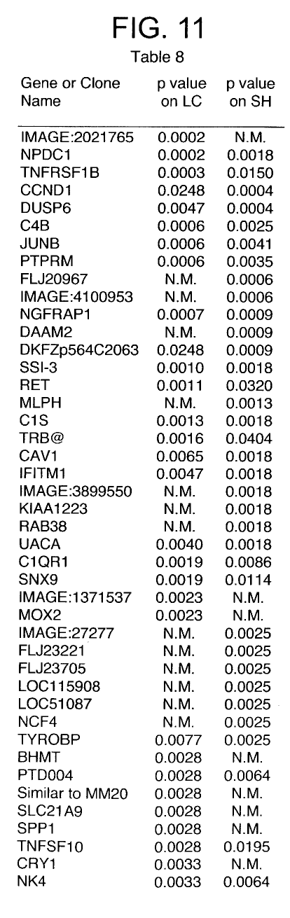

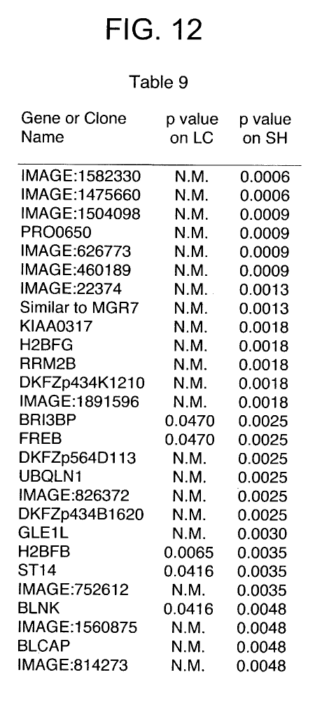

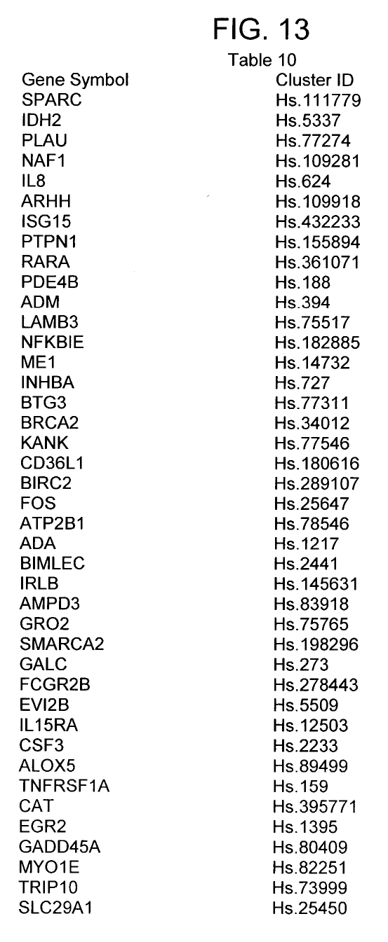

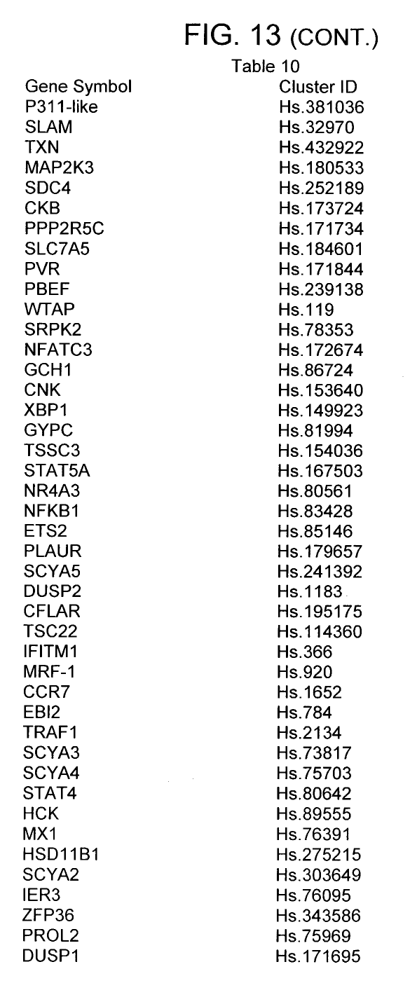

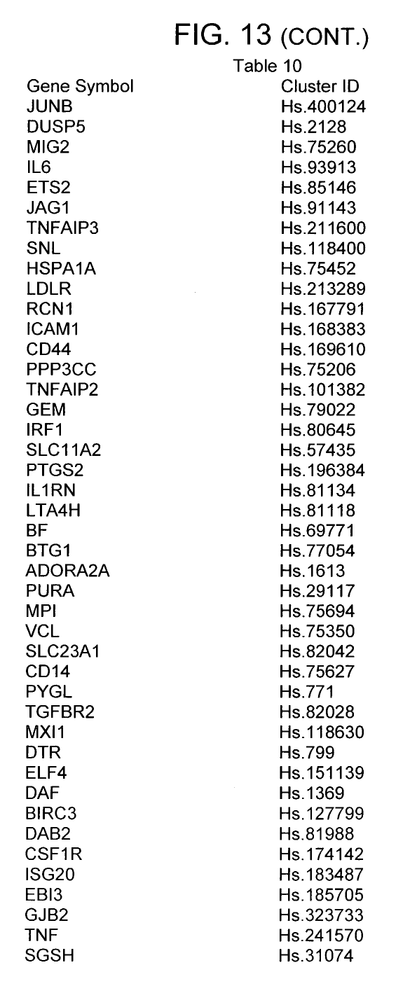

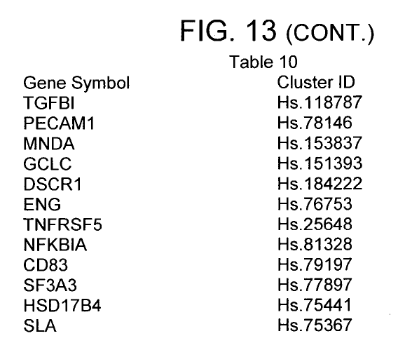

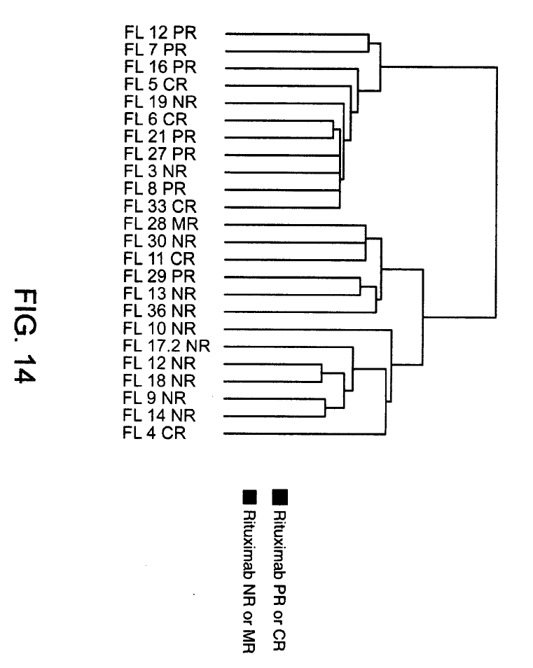

Representative genes/proteins of interest in certain embodiments include, but are not limited to, the genes/proteins provided in Table 1 (FIG. 4). Representative genes/proteins of interest in certain embodiments include, but are not limited to, the genes/proteins provided in Table 2 (FIG. 5). Representative genes/proteins of interest in certain embodiments include, but are not limited to, the genes/proteins provided in Table 3 (FIG. 6). Representative genes/proteins of interest in certain embodiments include, but are not limited to, the genes/proteins provided in Table 4 (FIG. 7). Representative genes/proteins of interest in certain embodiments include, but are not limited to, the genes/proteins provided in Table 5 (FIG. 8). Representative genes/proteins of interest in certain embodiments include, but are not limited to, the genes/proteins provided in Table 6 (FIG. 9). Representative genes/proteins of interest in certain embodiments include, but are not limited to, the genes/proteins provided in Table 7 (FIG. 10). Representative genes/proteins of interest in certain embodiments include, but are not limited to, the genes/proteins provided in Table 8 (FIG. 11). Representative genes/proteins of interest in certain embodiments include, but are not limited to, the genes/proteins provided in Table 9 (FIG. 12). Representative genes/proteins of interest in certain embodiments include, but are not limited to, the genes/proteins provided in Table 10 (FIG. 13). (Note that for all of the tables of genes appearing in the Figures to this application, the exact sequence of the clone identified in the table can be determined through the NCBI Entrez nucleotide database at located at the website produced by placing “http://www.” before “ncbi.nlm.nih.gov/entrez/guery.fcgi?CMD=search&db=nucleotide”; the sequence for a specific clone is then obtained by entering the clone ID in quotes as the search term).

In certain embodiments, at least one of the genes/proteins in the prepared expression profile is from one or more of Tables 1 to 10, where the expression profile may include expression data for 5, 10, 20, 50, 75 or more of, including all of, the genes/proteins listed in one or more of Tables 1 to 10. The number of different genes/proteins whose expression and/or quantity data, i.e., presence or absence of expression, as well as expression/quantity level, that are included in the expression profile that is generated may vary, but is typically at least 2, and in many embodiments ranges from 2 to about 100 or more, sometimes from 3 to about 75 or more, including from about 4 to about 70 or more.

In certain embodiments, the expression profile obtained is a genomic or nucleic acid expression profile, where the amount or level of one or more nucleic acids in the sample is determined. In these embodiments, the sample that is assayed to generate the expression profile employed in the diagnostic methods is one that is a nucleic acid sample. The nucleic acid sample includes a plurality or population of distinct nucleic acids that includes the expression information of the phenotype determinative genes of interest of the cell or tissue being diagnosed. The nucleic acid may include RNA or DNA nucleic acids, e.g., mRNA, cRNA, cDNA etc., so long as the sample retains the expression information of the host cell or tissue from which it is obtained. The sample may be prepared in a number of different ways, as is known in the art, e.g., by mRNA isolation from a cell, where the isolated mRNA is used as is, amplified, employed to prepare cDNA, cRNA, etc., as is known in the differential expression art. The sample is typically prepared from a cell or tissue harvested from a subject to be diagnosed, e.g., via biopsy of tissue, using standard protocols, where cell types or tissues from which such nucleic acids may be generated include any tissue in which the expression pattern of the to be determined phenotype exists, including, but not limited to, disease cells or tissue, etc.

The expression profile may be generated from the initial nucleic acid sample using any convenient protocol. While a variety of different manners of generating expression profiles are known, such as those employed in the field of differential gene expression analysis, one representative and convenient type of protocol for generating expression profiles is array-based gene expression profile generation protocols. Such applications are hybridization assays in which a nucleic acid that displays “probe” nucleic acids for each of the genes to be assayed/profiled in the profile to be generated is employed. In these assays, a sample of target nucleic acids is first prepared from the initial nucleic acid sample being assayed, where preparation may include labeling of the target nucleic acids with a label, e.g., a member of signal producing system. Following target nucleic acid sample preparation, the sample is contacted with the array under hybridization conditions, whereby complexes are formed between target nucleic acids that are complementary to probe sequences attached to the array surface. The presence of hybridized complexes is then detected, either qualitatively or quantitatively. Specific hybridization technology which may be practiced to generate the expression profiles employed in the subject methods includes the technology described in U.S. Pat. Nos. 5,143,854; 5,288,644; 5,324,633; 5,432,049; 5,470,710; 5,492,806; 5,503,980; 5,510,270; 5,525,464; 5,547,839; 5,580,732; 5,661,028; 5,800,992; the disclosures of which are herein incorporated by reference; as well as WO 95/21265; WO 96/31622; WO 97/10365; WO 97/27317; EP 373 203; and EP 785 280. In these methods, an array of “probe” nucleic acids that includes a probe for each of the phenotype determinative genes whose expression is being assayed is contacted with target nucleic acids as described above. Contact is carried out under hybridization conditions, e.g., stringent hybridization conditions as described above, and unbound nucleic acid is then removed. The resultant pattern of hybridized nucleic acid provides information regarding expression for each of the genes that have been probed, where the expression information is in terms of whether or not the gene is expressed and, typically, at what level, where the expression data, i.e., expression profile, may be both qualitative and quantitative.

Alternatively, non-array based methods for quantitating the levels of one or more nucleic acids in a sample may be employed, including quantitative PCR, and the like.

Where the expression profile is a protein expression profile, any convenient protein quantitation protocol may be employed, where the levels of one or more proteins in the assayed sample are determined. Representative methods include, but are not limited to: proteomic arrays, flow cytometry, standard immunoassays, etc.

Following obtainment of the expression profile from the sample being assayed, the expression profile is compared with a reference or control profile to make a diagnosis regarding the therapy responsive phenotype of the cell or tissue, and therefore host, from which the sample was obtained/derived. The terms “reference” and “control” as used herein mean a standardized pattern of gene expression or levels of expression of certain genes to be used to interpret the expression signature of a given patient and assign a prognostic class The reference or control profile may be a profile that is obtained from a cell/tissue known to have the desired phenotype, e.g., responsive phenotype, and therefore may be a positive reference or control profile. In addition, the reference/control profile may be from a cell/tissue known to not have the desired phenotype, and therefore be a negative reference/control profile.

In certain embodiments, the obtained expression profile is compared to a single reference/control profile to obtain information regarding the phenotype of the cell/tissue being assayed. In yet other embodiments, the obtained expression profile is compared to two or more different reference/control profiles to obtain more in depth information regarding the phenotype of the assayed cell/tissue. For example, the obtained expression profile may be compared to a positive and negative reference profile to obtain confirmed information regarding whether the cell/tissue has the phenotype of interest.

The comparison of the obtained expression profile and the one or more reference/control profiles may be performed using any convenient methodology, where a variety of methodologies are known to those of skill in the array art, e.g., by comparing digital images of the expression profiles, by comparing databases of expression data, etc. Patents describing ways of comparing expression profiles include, but are not limited to, U.S. Pat. Nos. 6,308,170 and 6,228,575, the disclosures of which are herein incorporated by reference. Methods of comparing expression profiles are also described above.

The comparison step results in information regarding how similar or dissimilar the obtained expression profile is to the control/reference profiles, which similarity/dissimilarity information is employed to determine the phenotype of the cell/tissue being assayed. For example, similarity with a positive control indicates that the assayed cell/tissue has a responsive phenotype. Likewise, similarity with a negative control indicates that the assayed cell/tissue has a non-responsive phenotype.

Depending on the type and nature of the reference/control profile(s) to which the obtained expression profile is compared, the above comparison step yields a variety of different types of information regarding the cell/tissue that is assayed. As such, the above comparison step can yield a positive/negative determination of a responsive phenotype of an assayed cell/tissue.

In many embodiments, the above-obtained information about the cell/tissue being assayed is employed to diagnose a host, subject or patient with respect to responsive to therapeutic antibody therapy, as described above.

The subject methods further find use in pharmacogenomic applications. In these applications, a subject/host/patient is first diagnosed for the presence of absence of a responsive phenotype using a protocol such as the diagnostic protocol described in the preceding section.

The subject is then treated using a pharmacological protocol, where the suitability of the protocol for a particular subject/patient is determined using the results of the diagnosis step. More specifically, where the identified phenotype is responsive, an appropriate therapeutic antibody treatment protocol is then employed to treat the patient. Alternatively, where a patient is identified as having a non-responsive phenotype, non-antibody protocols are then employed.

Databases of Expression Profiles of Phenotype Determinative Genes

Also provided are databases of expression profiles of phenotype determinative genes. Such databases will typically comprise expression profiles of various cells/tissues having responsive phenotypes, negative expression profiles, etc., where such profiles are further described below.

The expression profiles and databases thereof may be provided in a variety of media to facilitate their use. “Media” refers to a manufacture that contains the expression profile information of the present invention. The databases of the present invention can be recorded on computer readable media, e.g. any medium that can be read and accessed directly by a computer. Such media include, but are not limited to: magnetic storage media, such as floppy discs, hard disc storage medium, and magnetic tape; optical storage media such as CD-ROM; electrical storage media such as RAM and ROM; and hybrids of these categories such as magnetic/optical storage media. One of skill in the art can readily appreciate how any of the presently known computer readable mediums can be used to create a manufacture comprising a recording of the present database information. “Recorded” refers to a process for storing information on computer readable medium, using any such methods as known in the art. Any convenient data storage structure may be chosen, based on the means used to access the stored information. A variety of data processor programs and formats can be used for storage, e.g. word processing text file, database format, etc.

As used herein, “a computer-based system” refers to the hardware means, software means, and data storage means used to analyze the information of the present invention. The minimum hardware of the computer-based systems of the present invention comprises a central processing unit (CPU), input means, output means, and data storage means. A skilled artisan can readily appreciate that any one of the currently available computer-based system are suitable for use in the present invention. The data storage means may comprise any manufacture comprising a recording of the present information as described above, or a memory access means that can access such a manufacture.

A variety of structural formats for the input and output means can be used to input and output the information in the computer-based systems of the present invention. One format for an output means ranks expression profiles possessing varying degrees of similarity to a reference expression profile. Such presentation provides a skilled artisan with a ranking of similarities and identifies the degree of similarity contained in the test expression profile.

Reagents and Kits

Also provided are reagents and kits thereof for practicing one or more of the above-described methods. The subject reagents and kits thereof may vary greatly. Reagents of interest include reagents specifically designed for use in production of the above-described expression profiles of phenotype determinative genes.

One type of such reagent is an array of probe nucleic acids in which the phenotype determinative genes of interest are represented. A variety of different array formats are known in the art, with a wide variety of different probe structures, substrate compositions and attachment technologies. Representative array structures of interest include those described in U.S. Pat. Nos. 5,143,854; 5,288,644; 5,324,633; 5,432,049; 5,470,710; 5,492,806; 5,503,980; 5,510,270; 5,525,464; 5,547,839; 5,580,732; 5,661,028; 5,800,992; the disclosures of which are herein incorporated by reference; as well as WO 95/21265; WO 96/31622; WO 97/10365; WO 97/27317; EP 373 203; and EP 785 280.

In many embodiments, the arrays include probes for at least 1 of the genes listed in one or more of Tables 1 to 10. In certain embodiments, the number of genes that are from one or more of Tables 1 to 10 that is represented on the array is at least 5, at least 10, at least 25, at least 50, at least 75 or more, including all of the genes listed in one or more of Tables 1 to 10. The subject arrays may include only those genes that are listed in one or more of Tables 1-10, or they may include additional genes that are not listed in one or more of Tables 1 to 10. Where the subject arrays include probes for such additional genes, in certain embodiments the number % of additional genes that are represented does not exceed about 50%, usually does not exceed about 25%. In many embodiments where additional “non-Table 1-10” genes are included, a great majority of genes in the collection are phenotype determinative genes, where by great majority is meant at least about 75%, usually at least about 80% and sometimes at least about 85, 90, 95% or higher, including embodiments where 100% of the genes in the collection are phenotype determinative genes.

Another type of reagent that is specifically tailored for generating expression profiles of phenotype determinative genes is a collection of gene specific primers that is designed to selectively amplify such genes. Gene specific primers and methods for using the same are described in U.S. Pat. No. 5,994,076, the disclosure of which is herein incorporated by reference. Of particular interest are collections of gene specific primers that have primers for at least 1 of the genes listed in one or more of Tables 1 to 10, often a plurality of these genes, e.g., at least 2, 5, 10, 15 or more. In certain embodiments, the number of genes that are from Table 1 that have primers in the collection is at least 5, at least 10, at least 25, at least 50, at least 75 or more, including all of the genes listed in one or more of Tables 1 to 10. The subject gene specific primer collections may include only those genes that are listed in one or more of Tables 1 to 10, or they may include primers for additional genes that are not listed in one or more of Tables 1 to 10. Where the subject gene specific primer collections include primers for such additional genes, in certain embodiments the number % of additional genes that are represented does not exceed about 50%, usually does not exceed about 25%. In many embodiments where additional “non-Tables 1 to 10” genes are included, a great majority of genes in the collection are phenotype determinative genes, where by great majority is meant at least about 75%, usually at least about 80% and sometimes at least about 85, 90, 95% or higher, including embodiments where 100% of the genes in the collection are phenotype determinative genes.

The kits of the subject invention may include the above-described arrays and/or gene specific primer collections. The kits may further include one or more additional reagents employed in the various methods, such as primers for generating target nucleic acids, dNTPs and/or rNTPs, which may be either premixed or separate, one or more uniquely labeled dNTPs and/or rNTPs, such as biotinylated or Cy3 or Cy5 tagged dNTPs, gold or silver particles with different scattering spectra, or other post synthesis labeling reagent, such as chemically active derivatives of fluorescent dyes, enzymes, such as reverse transcriptases, DNA polymerases, RNA polymerases, and the like, various buffer mediums, e.g. hybridization and washing buffers, prefabricated probe arrays, labeled probe purification reagents and components, like spin columns, etc., signal generation and detection reagents, e.g. streptavidin-alkaline phosphatase conjugate, chemifluorescent or chemiluminescent substrate, and the like.

In addition to the above components, the subject kits will further include instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another means would be a computer readable medium, e.g., diskette, CD, etc., on which the information has been recorded. Yet another means that may be present is a website address which may be used via the internet to access the information at a removed site. Any convenient means may be present in the kits.

The following examples are offered by way of illustration and not by way of limitation.

### EXPERIMENTAL

I. Materials and Methods

A. Microarray Procedures

Freshly frozen lymph node samples were obtained after informed consent from patients who underwent excisional biopsy at Stanford University Medical Center (SUMC) and subsequently received rituximab. Tonsil and spleen samples were similarly obtained from patients treated at SUMC. Biopsy samples were stored frozen in optimal cutting temperature medium. Poly-(A)⁺ mRNA was obtained from biopsy samples after homogenization of tissue using the FastTrack 2.0 kit (Invitrogen). An experimental cDNA probe incorporating Cy5 dye was generated from mRNA from malignant and normal lymphoid tissues; a common reference cDNA probe incorporating Cy3 dye was from mRNA derived from a panel of cell lines and probes were hybridized to cDNA microarrays as previously described (Perou et al., Nature (2000) 406, 747-52; Alizadeh et al., Nature (2000) 403, 503-11).

B. Statistical Analysis

Prior to statistical analysis, individual data points were filtered for data quality and adequate signal intensity. Hierarchical cluster analysis has previously been described (Eisen et al., Proc Natl Acad Sci U S A (1998) 95, 14863-8). Prior to supervised statistical analysis, values for missing data were estimated and singular value decomposition (SVD) was used to filter data for a pattern corresponding to a systematic, date specific difference in array processing (Alter et al., Proc Natl Acad Sci U S A (2000) 97, 10101-6). Wilcoxon rank sum test was used to generate a rank list of genes whose corresponding mRNA levels differ significantly in rituximab responders versus non-responders (0. Troyanskaya et al, Bioinformatics (2002) 18:1454-1461). Also used was the Statistical Analysis for Microarrays (SAM) see Tusher et al. PNAS (2001) 98:10515.

C. Flow Cytometry

Cells from lymph node biopsies were manipulated into single cell suspensions and viable mononuclear cells were isolated by centrifugation in a Ficoll-Paque gradient then washed. Cells were then stained with single fluorochrome conjugated antibodies anti-CD3, anti-CD4, anti-CD8, anti-CD19 and/or anti-CD20, and anti-CD37 from Becton Dickinson Immunocytometry Systems.

II. Results

A. Patient Characteristics

Tumor samples from 24 patients were analyzed in our study. The following criteria were required for patient tumor samples to be included in cDNA microarray analysis; freshly frozen lymph node biopsy material was obtained prior to any systemic therapy, pathology was interpreted as follicular NHL (follicular small cleaved, follicular mixed or follicular large cell histology), the patient subsequently received rituximab treatment with documentation of objective response and mRNA yield was adequate to allow cDNA microarray analysis. In all cases, biopsy and pathology review were performed at Stanford University Medical Center. Rituximab treatment was given at SUMC or by an outside oncologist. Patient characteristics are summarized below.

| | NR/MR | PR/CR |
|---|---|---|
| Patient Characteristics | (n = 12) | (n = 12) |
| Sex (M:F) | 10:2 | 7:5 |
| Age at rituximab treatment ± SD | 51.6 ± 9.2 | 52.2 ± 13.7 |
| Pathology | | |
| FSC | 9 | 7 |
| FM | 2 | 5 |
| FLC | 1 | 0 |
| Mean no. of courses prior | 2.42 | 2.42 |
| chemotherapy | | |
| Prior high dose chemotherapy | 3 | 2 |

Patient characteristics by response to rituximab treatment. NR indicates no response; MR, minimally response; PR, partial response; CR, complete response; FSC, follicular small cleaved; FM, follicular mixed; FLC, follicular large cell. Age data are presented as mean age in years for each group±standard deviation.

No significant differences were observed between responders and non-responders in age or treatment history (see above). All patients received, except one, at least one course of chemotherapy prior to receiving rituximab (range 0 to 6 prior courses). One patient in the CR/PR group received a shared anti-idiotype antibody as the sole prior treatment. For the treatment of FL, a course of rituximab typically consists of four weekly infusions of 375 mg/m²; 21 of 24 patients received this dosing regimen, including all of the patients in the non-responder (NR) group. In the PR/CR group, two patients received a single dose of 375 mg/m² and one patient received eight weekly doses of 375 mg/m² with a documented near CR at six weeks of treatment. Five patients achieved CR in response to rituximab, seven experienced PR and 12 patients had no or minimal response. The overall response rate (CR+PR) in this cohort was 50% which is similar to the overall response rate of 60% demonstrated for FL in the pivotal Phase II trial.

B. Gene Expression Patterns from Rituximab Non-Responders Show Similarity to Normal Lymphoid Tissue

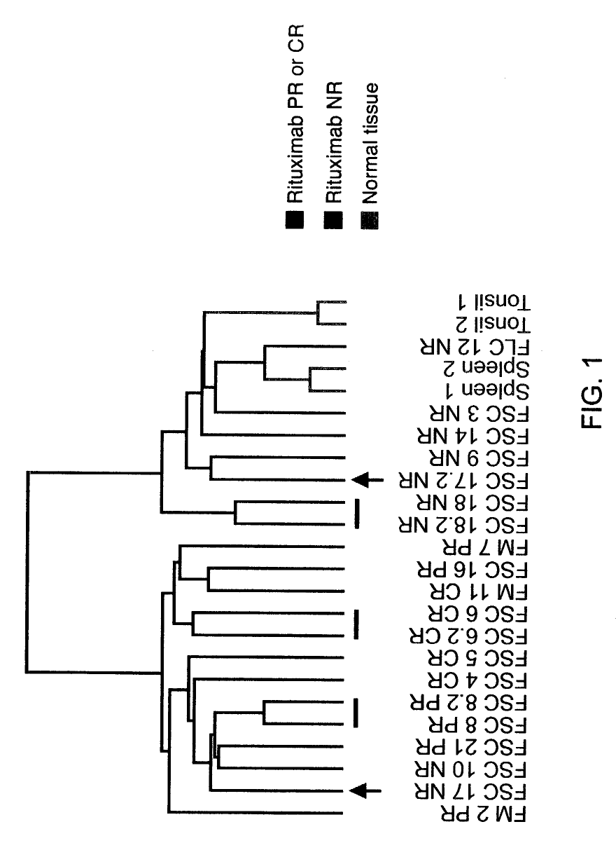

An overview of the gene expression patterns from FL patients was generated by hierarchical cluster analysis of data from 16 FL with samples from non-malignant tonsil and spleen (FIG. 1). The hierarchical cluster algorithm employed arranges genes and tissue samples based on the degree of similarity in the gene expression pattern (Eisen et al., Proc. Nat'l Acad. Sci. USA (1998) 95:14863-14868). In addition to tumor material obtained prior to treatment, we included tissue samples from later biopsies for four patients for whom such material was available. These samples were obtained 2.5 to 6.5 years after the initial biopsy. In all cases, patients received one or more courses of chemotherapy between the initial and later biopsies and the histopathological diagnosis was the same for both biopsy samples.

Gene expression data was derived from microarrays containing over 43,000 spotted cDNAs (the Stanford Human or SH array). Prior to clustering, data were median centered and filtered for data quality and signal intensity above background. To select genes differentially expressed in the samples, a variance filter was used such that genes were included only if a two-fold difference in normalized expression was observed for at least three arrays. After filters were applied, expression data from 2,394 cDNAs, representing 2,037 unique genes, were used for analysis. Interestingly, the dendrogram shown in FIG. 1 shows that a subset of the FL samples exhibit a gene expression pattern more similar to non-malignant lymphoid tissues than to other FL samples. The FL samples from rituximab non-responders clustered disproportionately with normal tonsil and spleen samples (P<0.005, Fisher's exact test). This shows that difference in gene expression may be used to predict the outcome of rituximab treatment in FL patients.

Several features of the dendrogram in FIG. 1 indicate that the clustering of tissue samples based on gene expression patterns is reflective of biologically relevant similarities between samples. First, pairs of normal spleen and tonsil tissue cluster very closely together. Second, FL lymph node biopsies from the same individual cluster together in three of the four cases (see black bars in FIG. 1) despite the passage of time and treatment with systemic chemotherapy between biopsies. The two samples obtained from patient 17 do not cluster together (FIG. 1 black arrows) with the pretreatment biopsy clustering on the responders portion of the dendrogram and the later sample displaying a gene expression pattern more similar to that of rituximab non-responders and normal lymphoid tissue. The latter sample was predictive of the patient's actual outcome after rituximab treatment; the patient did not respond. This finding suggests that the gene expression phenotype of FL may change over time.

C. Genes with Increased Expression in Rituximab Non-Responders

To better understand differences in gene expression in FL lymph nodes form rituximab responders versus non-responders, we used supervised statistical methods to determine which genes had the most significant differences in expression in the two rituximab response groups. We measured gene expression from 24 independent patient samples, 12 from rituximab responders and 12 from rituximab non-responders; the clinical characteristics of these patients are summarized in Table 2. Unfortunately, technical limitations prevented characterization of all of the samples on the array type used to generate the overview of gene expression thus we measured expression levels on a microarray derived from the Lymphochip, a microarray enriched for genes expressed in lymphoid cells or known to be involved in immunology or cancer as previously described (Rosenwald et al., J Exp Med (2001) 194, 1639-47; Alizadeh et al., Cold Spring Harb Symp Quant Biol (1999) 64, 71-8).

A rank list of expression levels of all well-measured genes was generated using the Wilcoxon rank sum test. A list of genes whose expression differed between responders and non-responders with a p value less than or equal to 0.004 was chosen for further analysis. Data for a given cDNA was included in this analysis if data quality and signal intensity criteria were met; no variance filter was applied. The p value cutoff of 0.004 was chosen based on an assay of data robustness which involved leaving out data from one, two or three patient samples at random then recalculating the p value for the remaining samples; please see web supplement for details. A comparison of various non-parametric statistical methods for the analysis of complex gene expression data has demonstrated that Wilcoxon analysis is relatively conservative in terms of the significance assigned.

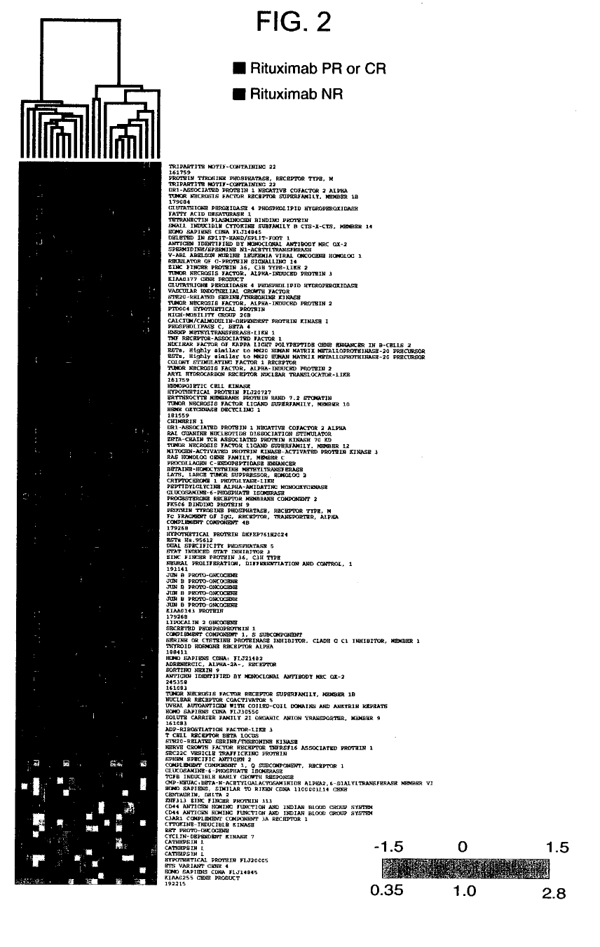

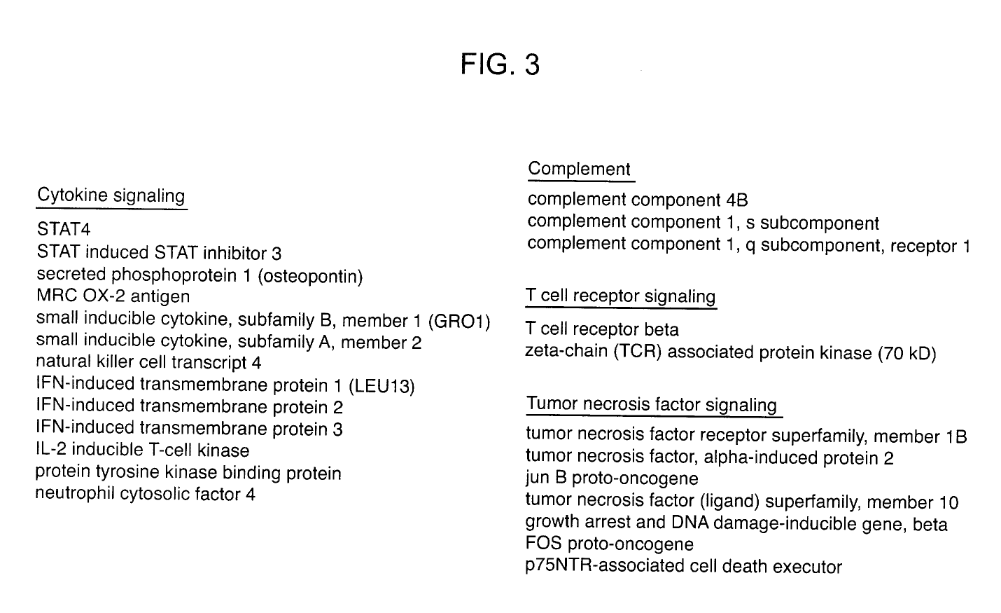

This analysis generated a list of 111 cDNAs, representing 104 independent genes (provided below in Table 3), with significantly increased expression in non-responders relative to responders. Expression data for these genes are represented visually in the cluster presented in FIG. 2. Examination of the known functions of these genes indicates that many of the gene products are functionally related as outlined in FIG. 3. The biological processes represented in this gene list suggest that there is a greater diversity of cell types contained in lymph nodes of rituximab non-responders and/or the signaling state of these cells is significantly different. Genes expressed in greater abundance in non-responders suggest an increase in T-cell activity as well and other effectors of cell mediated immunity. Indeed, this diversity of transcripts represented may explain the similarity between rituximab non-responders and normal lymphoid tissues, seen in FIG. 1.

Table 3 appearing in FIG. 6 provides a list of genes displaying higher mRNA levels in rituximab non-responders as identified by Wilcoxon rank sum test with p<0.004.

Using the same criteria, only 30 cDNAs, representing 30 genes, were identified as upregulated in responders relative to non-responders. These genes are listed in Table 4 appearing in FIG. 7.

Of these, over half are unnamed genes and there is no apparent biological relationship among the genes listed. This finding raises the possibility that the list generated does not represent a true difference between responders and non-responders.

Using another statistical method, significance analysis for microarrays (SAM), 147 genes (provided in Table 5 appearing in FIG. 8) are identified as being significantly overexpressed in rituximab non-responders with a median false discovery rate of slightly more than 20%. In contrast, no genes were identified as having significantly increased expression on the responding group. The similarity between the genelists is remarkable with over 60% of the genes identified by SAM also represented on the Wilcoxon rank list (Tusher et al., Proc Natl Acad Sci U S A (2001) 98, 5116-21).

Additional lists of genes of interest identified using various approaches includes those appearing in: FIG. 9, Table 6; FIG. 10, Table 7; FIG. 11, Table 8; FIG. 12, Table 9; and FIG. 13, Table 10.

D. Genes Increased in non-Responders are Involved in Inflammation and Cell-Mediated Immunity

With the goal of better understanding the biological differences between rituximab responders and non-responders, the current scientific literature for all 104 genes identified in the above analysis was reviewed. Genes were categorized by biological function and several themes emerged from this analysis. Many of these genes are involved in the cellular immune response and inflammation, specifically cytokine and tumor necrosis factor signaling (FIG. 3). Also present are the T-cell receptor beta and the TCR associated protein ZAP70. These finding suggest that mediators of cellular immune response, such T-cells, macrophages, monocytes, and NK cells, may be relatively more abundant and/or more activated in lymph node tissue of rituximab non-responders. Several mRNAs for proteins involved in the complement cascade are more abundant in non-responders, including complement components, a regulator of complement acitivation, and complement receptors. In many cases, experimental evidence exists to justify inclusion of these genes into more than one of the listed categories. Furthermore, several genes that were not listed have been shown to play some less well-defined role in inflammation.

E. Flow Cytometry Confirms Difference in the Cellular Composition of Lymph Node from Rituximab Non-Responders

The results of the gene expression analysis described predict that difference in mRNA abundance for particular genes between rituximab responders and non-responders may be, at least in part, a reflection of differences in the cellular composition of lymph nodes form these two groups. To test this hypothesis, we compared the cellular composition of FL lymph nodes from 36 patients who were treated with rituximab, 23 responders and 13 non-responders. Patients were included in this analysis if flow cytometry data from a biopsy specimen was available and rituximab treatment and outcome were documented; data from 36 patients was available (13 NR and 23 PR/CR). Biopsy samples from 20 patients were analyzed both by flow cytometry and cDNA microarray analysis (above). The percentage of T cells (CD3+), T cell subsets (CD4+ and CD8+), B cells (CD19+ and/or CD20+) and activated B cells (CD37+) in a cell suspension derived from lymph node was assessed by flow cytometry. The results of this analysis, as summarized in Table 6, show that lymph nodes from rituximab non-responders have a lower percentage of B-cells than those from rituximab responders. While differences in the mean percentage of T-cells and T-cell subsets do not achieve statistical significance, it is likely that the lymph nodes from non-responders have a higher percentage of non-B-cells on the average.

| | NR/MR | PR/CR | |
|---|---|---|---|
| Antigen | (n = 12) | (n = 12) | p value |
| CD3 | 28.6 ± 8.0 | 20.8 ± 3.9 | 0.134 |
| CD4 | 22.3 ± 6.0 | 14.6 ± 3.0 | 0.056 |
| CD8 | 3.8 ± 1.3 | 4.1 ± 1.3 | 0.808 |
| CD37 | 69.9 ± 7.2 | 80.9 ± 3.4 | 0.024 |
| CD19/CD20 | 67.6 ± 6.1 | 77.9 ± 3.9 | 0.013 |

Percentage of lymph node cells that are CD3, CD4, CD8, CD37 and CD19/20 positive. Data are expressed as mean ± 95% confidence interval. Data was analyzed by two-sided t-test and differences are considered statistically significant if p < 0.05.

It is evident that subject invention provides a convenient and effective way of determining whether a patient will be responsive to antineoplastic, e.g., antibody, therapy. The subject methods will provide a number of benefits, including avoidance of delays in alternative treatments, elimination of exposure to adverse effects of therapeutic antibodies and reduction of unnecessary expense. As such, the subject invention represents a significant contribution to the art.

All publications and patent applications cited in this specification are herein incorporated by reference as if each individual publication or patent application were specifically and individually indicated to be incorporated by reference. The citation of any publication is for its disclosure prior to the filing date and should not be construed as an admission that the present invention is not entitled to antedate such publication by virtue of prior invention.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

What is claimed is:

1. A method of determining whether a subject suffering from a neoplastic disease is responsive to antineoplastic therapy, said method comprising:
(a) obtaining an expression profile for a sample from said subject; and
(b) comparing said obtained expression profile to a control expression profile to determine whether said subject is responsive to said antineoplastic therapy.

2. The method according to claim 1, wherein said neoplastic disease is non-Hodgkin's lymphoma (NHL).

3. The method according to claim 2, wherein said NHL is follicular lymphoma.

4. The method according to claim 1, wherein said antineoplastic therapy is antibody therapy.

5. The method according to claim 4, wherein said antibody therapy comprises use of a therapeutic antibody or mimetic thereof that specifically binds to cd20.

6. The method according to claim 5, wherein said therapeutic antibody is a monoclonal antibody.

7. The method according to claim 6, wherein said monoclonal antibody is Rituximab.

8. The method according to claim 1, wherein said expression profile is determined using a microarray.

9. A method of treating a subject suffering from a neoplastic disease, said method comprising:
(a) determining whether said subject is responsive to antineoplastic therapy by:
(i) obtaining an expression profile for a sample from said subject; and
(ii) comparing said obtained expression profile to a reference expression profile to determine whether said subject is responsive to said antineoplastic therapy;
(b) determining a treatment protocol based on said determination of step (a); and
(c) treating said host according to said determined treatment protocol of step (b).

10. The method according to claim 9, wherein said neoplastic disease is non-Hodgkin's lymphoma (NHL).

11. The method according to claim 10, wherein said NHL is follicular lymphoma.

12. The method according to claim 9, wherein said antineoplastic therapy is antibody therapy.

13. The method according to claim 12, wherein said antibody therapy comprises use of a therapeutic antibody or mimetic thereof that specifically binds to cd20.

14. The method according to claim 13, wherein said therapeutic antibody is a monoclonal antibody.

15. The method according to claim 14, wherein said monoclonal antibody is Rituximab.

16. The method according to claim 9, wherein said expression profile is determined using a microarray.

17. A reference expression profile for a neoplastic phenotype that is one of:
(a) responsive to antineoplastic therapy; or
(b) non-responsive to antineoplastic therapy;
wherein said expression profile is recorded on a computer readable medium.

18. The reference expression profile according to claim 17, wherein said neoplastic phenotype is a non-Hodgkin's lymphoma (NHL) phenotype.

19. The expression profile according to claim 17, wherein said expression profile includes at least one of the genes from Tables 1 to 10.

20. The expression profile according to claim 17, wherein said expression profile is a profile for a phenotype that is responsive to antibody therapy.

21. The expression profile according to claim 17, wherein said expression profile is a profile for a phenotype that is non-responsive to antibody therapy.

22. The expression profile according to claim 18, wherein said NHL is follicular lymphoma.

23. The expression profil according to claim 17, wherein said antineoplastic therapy is antibody therapy.

24. The expression profile according to claim 23, wherein said antibody therapy comprises use of a therapeutic antibody or mimetic thereof that specifically binds to cd20.

25. The expression profile according to claim 24, wherein said therapeutic antibody is a monoclonal antibody.

26. The expression profile according to claim 25, wherein said therapeutic antibody is Rituximab.

27. A collection of gene specific primers, said collection comprising:
gene specific primers specific for at least two of the genes of Tables 1 to 10.

28. The collection according to claim 27, wherein said collection comprises at least 10 gene specific primers.

29. A array of probe nucleic acids immobilized on a solid support, said array comprising:
a plurality of probe nucleic acid compositions, wherein each probe nucleic acid composition is specific for a gene whose expression profile is indicative of a NHL therapeutic antibody responsive phenotype, wherein at least two of said probe nucleic acid compositions correspond to genes listed in Tables 1 to 10.

30. A kit for use in determining the neoplastic disease therapeutic antibody responsive phenotype of a source of a nucleic acid sample, said kit comprising:
at least one of:
(a) an array according to claim 29; and
(b) a collection of gene specific primers according to claim 27.

31. The kit according to claim 30, wherein said kit comprises both said array and said collection of gene specific primers.

32. A method of determining whether a subject suffering from a neoplastic disease is responsive to antineoplastic therapy, said method comprising:
(a) obtaining an cell mediated immunity profile for said subject; and
(b) using said obtained cell mediated immunity profile to determine whether said subject is responsive to said antineoplastic therapy.

33. The method according to claim 32, wherein said antineoplastic therapy is antibody therapy.

34. The method according to claim 32, wherein said cell mediated immunity profile is an expression profile.
